(19) United States
(12) Patent Application Publication
(10) Pub. No.: US 2026/0226106 A1
(43) Pub. Date: Aug. 6, 2026

(54) NEW PROCESS FOR THE PREPARATION OF AMORPHOUS VOCLOSPORIN

(71) Applicant: Indena S.P.A., Milano (IT)

(72) Inventors: Fabio Morana, Novara (IT); Davide Botteri, Pavia (IT); Jacopo Roletto, Torino (IT); Paolo Paissoni, Druento (TO) (IT)

(73) Assignee: Indena S.P.A., Milano (IT)

(21) Appl. No.: 19/150,553

(22) PCT Filed: Jan. 24, 2024
(86) PCT No.: PCT/IB2024/050667

§ 371 Date: Jul. 24, 2025

(30) Foreign Application Priority Data
Jan. 25, 2023 (IT) 102023000001056

(51) Int. Cl. C07K 7/64

(52) U.S. Cl. CPC C07K 7/645

(57) ABSTRACT

The present invention concerns a new process for the preparation of voclosporin in amorphous form.

### FIELD OF THE INVENTION

The present invention concerns a new process for the preparation of amorphous voclosporin.

### BACKGROUND OF THE INVENTION

Voclosporin is a compound of formula I and analogue of cyclosporine A:

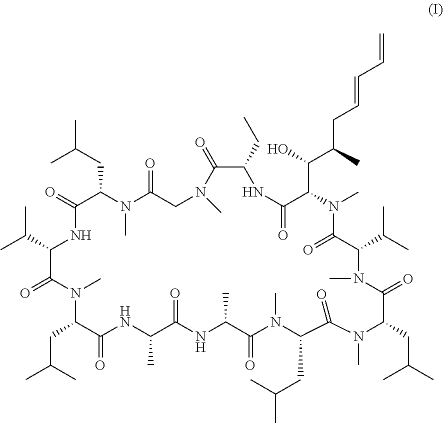 [SMILES: C=C/C=C/C[C@@H](C)[C@@H](O)[C@H]1C(=O)N[C@@H](CC)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

It is a calcineurin inhibitor that is used in pharmaceuticals and specifically as an active ingredient with an immunosuppressive effect, especially in the treatment of certain forms of lupus.

Although the compound itself has been known for some time, the Applicant has found that the methods of synthesis of voclosporin known to date have technological limitations related to their industrial applicability and scalability with particular reference to the possibility of obtaining the product with a high degree of purity in high yields and at low cost.

The Applicant noted in particular that the syntheses known to date frequently start from cyclosporine A, a product of natural origin also having an immunosuppressive effect, and subject said cyclosporine to a series of reactions involving the preparation of various intermediates, including acetyl cyclosporine A aldehyde and acetyl voclosporin, two compounds of formula III and IV respectively:

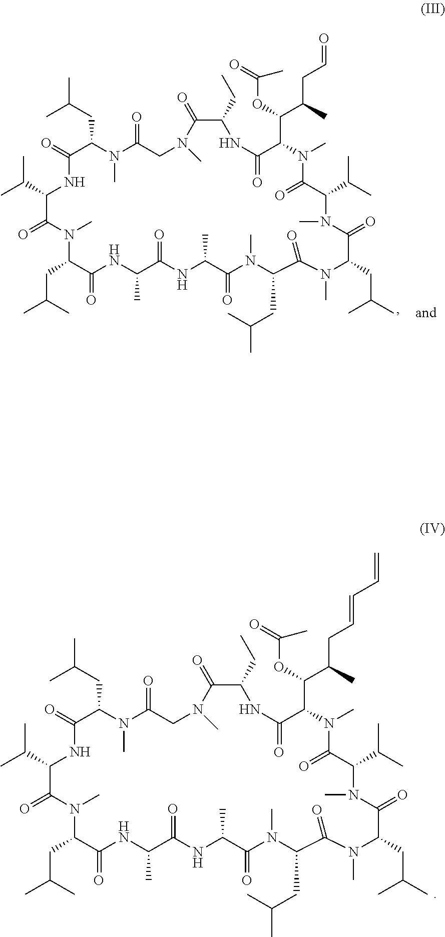

In this regard, the Applicant noted that the synthesis steps concerning these intermediate products, as well as the form and ease with which the final voclosporin is obtained, are particularly critical precisely in order to provide a synthesis process that allows high yields to be achieved at low cost and a product with a high degree of purity.

### SUMMARY OF THE INVENTION

The aim of the present invention is therefore to provide a new process for the synthesis of voclosporin that overcomes the current difficulties and limits, so as to provide a new process that allows high yields to be achieved at low cost and a product with a high degree of purity.

In accordance with the present invention, the Applicant has surprisingly found that it is possible to pursue the above-mentioned purpose by preparing voclosporin from cyclosporine A and by using special reaction conditions and expedients in the steps related to obtaining acetyl cyclosporine A, acetyl cyclosporine A aldehyde and acetyl voclosporin.

In particular, the Applicant discovered the possibility of obtaining intermediates in a simple and high-yield manner and also of obtaining the final product, voclosporin, in an amorphous form, which turned out to be a solid form particularly easy to handle and stable over time.

The process according to the present invention is therefore more competitive than existing processes for the synthesis of voclosporin, offering an industrially scalable alternative that allows it to be obtained in high yield and in a form that is easy to handle and stable over time.

Therefore, the present invention relates in its first aspect to a process for the preparation of voclosporin of formula I in amorphous form

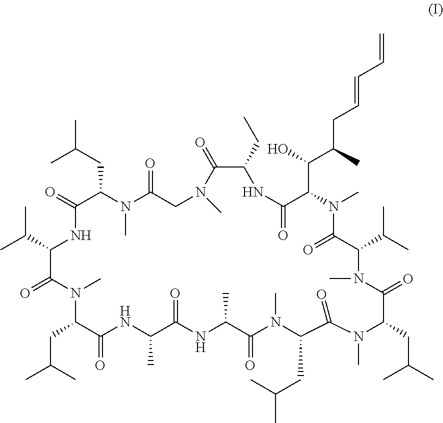 [SMILES: C=C/C=C/C[C@@H](C)[C@@H](O)[C@H]1C(=O)N[C@@H](CC)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

comprising the steps of:
a. acetylating cyclosporine A in the presence of methyl tert-butyl ether (MTBE) with acetic anhydride in a stoichiometric amount with respect to cyclosporine A, thus obtaining acetyl cyclosporine A of formula II:

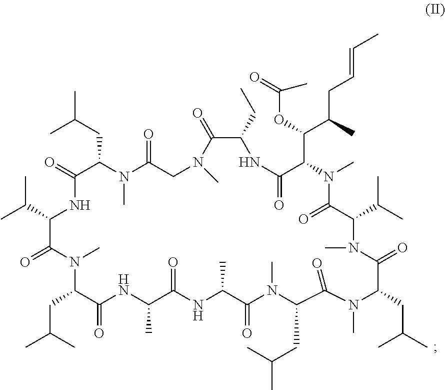 [SMILES: C/C=C/C[C@@H](C)[C@@H](OC(C)=O)[C@H]1C(=O)N[C@@H](CC)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

b. oxidising the acetyl cyclosporine A of formula II with at least one oxidising agent that is an osmiate salt in the presence of a base selected from the group consisting of dimethylpyridine (or lutidine) and 2,4,6-trimethylpyridine (or sym-collidine), wherein said base is in an amount such that a pH value in the range from 6 to 8 is achieved, and in the presence of a co-oxidant selected from the group consisting of a periodate of an alkali or alkaline earth metal and a hypochlorite of an alkali or alkaline earth metal, thus obtaining acetyl cyclosporine A aldehyde of formula III:

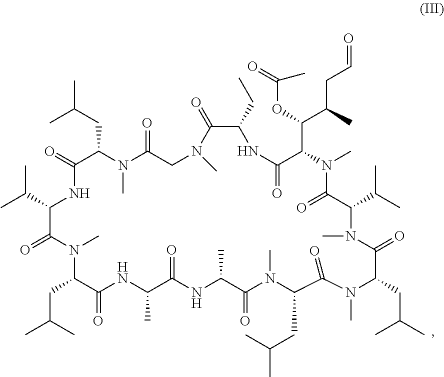 [SMILES: CC[C@@H]1NC(=O)[C@H]([C@H](OC(C)=O)[C@H](C)CC=O)N(C)C(=O)[C@H](C(C)C)N(C)C(=O)[C@H](CC(C)C)N(C)C(=O)[C@H](CC(C)C)N(C)C(=O)[C@@H](C)NC(=O)[C@H](C)NC(=O)[C@H](CC(C)C)N(C)C(=O)[C@H](C(C)C)NC(=O)[C@H](CC(C)C)N(C)C(=O)CN(C)C1=O]

c. converting the acetyl cyclosporine A aldehyde of formula III into acetyl voclosporin of formula IV:

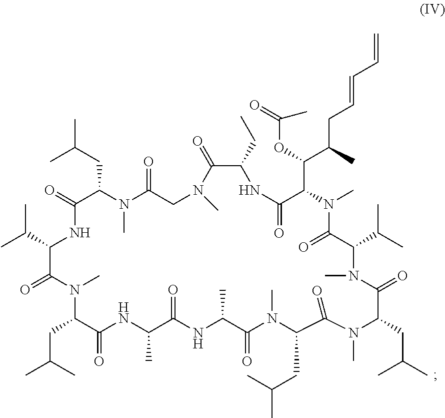 [SMILES: C=C/C=C/C[C@@H](C)[C@@H](OC(C)=O)[C@H]1C(=O)N[C@@H](CC)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

d. crystallising the acetyl voclosporin of formula IV with a solvent system selected from the group consisting of THF/n-heptane and MTBE, thus obtaining acetyl voclosporin in crystalline form;
e. hydrolysing the acetyl voclosporin of formula IV, thus obtaining voclosporin of formula l; and
f. isolating the voclosporin of formula I obtained in step e. obtaining said voclosporin of formula I in amorphous form, wherein said isolation is carried out by precipitation with a solvent/anti-solvent system, wherein said solvent is selected from the group consisting of MTBE, THF, 2-methyl THF, diisopropyl ether, acetonitrile, acetone, ethanol and cyclopentylmethylether, and said anti-solvent is a hydrocarbon selected from the group consisting of normal heptane, cyclohexane and pentane.

In fact, it was surprisingly discovered that the use of MTBE with acetic anhydride in stoichiometric amounts in step a. made it possible to avoid large consumption of acetic anhydride, which is a toxic reagent with a high environmental impact, to improve the reaction yield and to improve purity with respect to the process applied in the prior art.

Furthermore, advantageously and surprisingly, by using an osmiate salt as the oxidising agent in step b. and a co-oxidant selected from the group consisting of a periodate of an alkali or alkaline earth metal and a hypochlorite of an alkali or alkaline earth metal, the oxidation reaction of acetyl cyclosporine A to obtain acetyl cyclosporine A aldehyde was particularly advantageous in terms of yield and conduction simplicity. The presence of the base selected from the group consisting of dimethylpyridine (or lutidine) and 2,4,6-trimethylpyridine (or sym-collidine) advantageously allowed the carboxylic acid impurity to be suppressed, as will become clear from the experimental part.

It should also be noted that, thanks to the use of a solvent system selected from the group consisting of THF/n-heptane and MTBE for the isolation of acetyl voclosporin, it was also advantageously possible to obtain this intermediate product in crystalline form. The crystalline acetyl voclosporin of the invention exhibits a X-ray powder diffraction spectrum (XRPD), with peaks at characteristic angle values 2ϑ(±0.2) of 5.0, 5.5, 7.0, 8.5, 8.8, 10.2, 12.4, 16.5, 17.2 and 20.9.

Furthermore, the Applicant has surprisingly discovered that by precipitating voclosporin with a solvent/antisolvent system, wherein said solvent is selected from the group consisting of MTBE, THF, 2-methyl THF, diisopropyl ether, acetonitrile, acetone ethanol and cyclopentylmethylether, and said antisolvent is a hydrocarbon selected from the group consisting of normal heptane, cyclohexane and pentane, it is possible to obtain an amorphous form of the product characterised by high long-term stability and easy handling due to its powdery and non-sticky nature.

Depending on the various improvements made in the different synthesis steps starting from cyclosporine A and leading to the preparation of voclosporin, the present invention also has certain ancillary aspects.

In a further aspect, specifically, the present invention concerns a process for the preparation of acetyl cyclosporine A aldehyde of formula III:

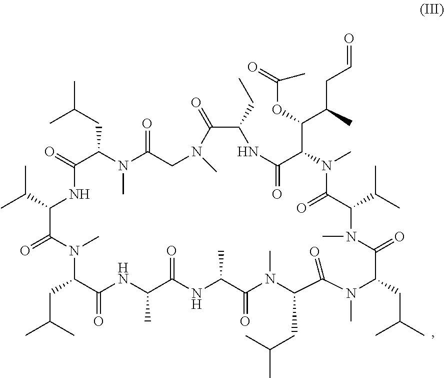 [SMILES: CC[C@@H]1NC(=O)[C@H]([C@H](OC(C)=O)[C@H](C)CC=O)N(C)C(=O)[C@H](C(C)C)N(C)C(=O)[C@H](CC(C)C)N(C)C(=O)[C@H](CC(C)C)N(C)C(=O)[C@@H](C)NC(=O)[C@H](C)NC(=O)[C@H](CC(C)C)N(C)C(=O)[C@H](C(C)C)NC(=O)[C@H](CC(C)C)N(C)C(=O)CN(C)C1=O]

comprising the steps of:
A. acetylating cyclosporine A in the presence of methyl tert-butyl ether (MTBE) with acetic anhydride in a stoichiometric amount with respect to cyclosporine A, thus obtaining acetyl cyclosporine A of formula II:

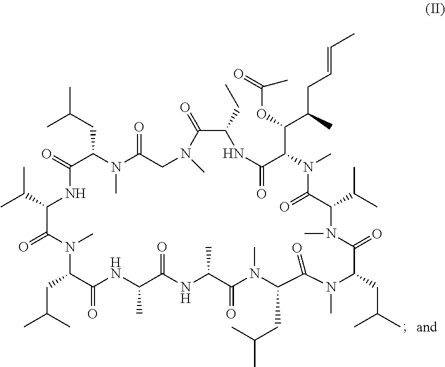 [SMILES: C/C=C/C[C@@H](C)[C@@H](OC(C)=O)[C@H]1C(=O)N[C@@H](CC)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

B. oxidising the acetyl cyclosporine A of formula II with at least one oxidising agent that is an osmiate salt in the presence of a base selected from the group consisting of dimethylpyridine (or lutidine) and 2,4,6-trimethylpyridine (or sym-collidine), wherein said base is in an amount such that a pH value in the range from 6 to 8 is achieved, and in the presence of a co-oxidant selected from the group consisting of a periodate of an alkali or alkaline earth metal and a hypochlorite of an alkali or alkaline earth metal, thus obtaining acetyl cyclosporine A aldehyde of formula III.

Advantageously, the acetyl cyclosporine A of formula II was obtained in crystalline form using the MTBE/n-heptane solvent system and exhibits a X-ray powder diffraction spectrum (XRPD), with peaks at characteristic angle values 2ϑ(±0.2) of 8.3, 9.1, 11.1, 11.3, 13.0, 15.0, 17.0, 17.6, 17.8, 19.1 and 19.6. Advantageously, the acetyl cyclosporine A of formula II was obtained in crystalline form using the MTBE/cyclohexane solvent system and exhibits a X-ray powder diffraction spectrum (XRPD), with peaks at characteristic angle values 2ϑ(±0.2) of 5.1, 5.4, 5.5, 9.0, 9.5, 10.2, 15.5, 17.4, 20.2 and 20.9.

The advantageousness in terms of yield and conduction simplicity of the preparation of the intermediate product acetyl cyclosporine A, advantageously in the described crystalline forms, and of the product acetyl cyclosporin aldehyde, which derives from the characteristics of step a. and step b., is indeed an advantage for the preparation of voclosporin, as it contributes substantially and even decisively to obtaining the desired product with high yield and under industrially scalable conditions. The Applicant has therefore found that this further aspect of the invention included in its first and principal aspect independently represents a key innovative and advantageous aspect in the context of the processes for obtaining voclosporin.

In further aspects thereof, the present invention also relates to a process for obtaining acetyl voclosporin of formula IV in crystalline form and to the acetyl voclosporin in crystalline form thus obtained.

The present invention therefore also relates to a process for obtaining acetyl voclosporin of formula IV in crystalline form

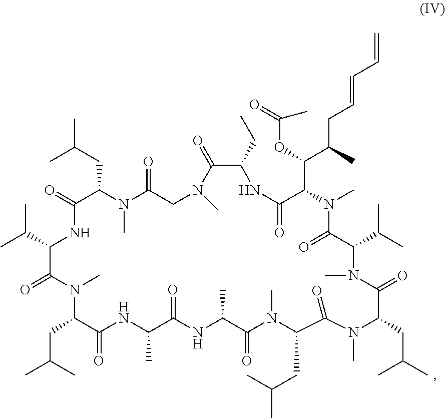 [SMILES: C=C/C=C/C[C@@H](C)[C@@H](OC(C)=O)[C@H]1C(=O)N[C@@H](CC)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

comprising the steps of:
   preparing acetyl voclosporin of formula IV, for example but not exclusively according to steps a. to c. of the process according to the first aspect of the invention;
   crystallising said acetyl voclosporin of formula IV with a solvent system selected from the group consisting of THF/n-heptane and MTBE, thus obtaining acetyl voclosporin in crystalline form.

Furthermore, the present invention relates to the new crystalline form of acetyl voclosporin thus obtainable and in particular to a crystalline form of acetyl voclosporin of the invention having a X-ray powder diffraction spectrum (XRPD) with peaks at the characteristic angle values 2ϑ(±0.2) of 5.0, 5.5, 7.0, 8.5, 8.8, 10.2, 12.4, 16.5, 17.2 and 20.9.

The possibility of obtaining acetyl voclosporin in crystalline form also represents an advantage within the process according to the first and main aspect of the invention but also, independently, a key innovative and advantageous aspect in the context of the processes for obtaining voclosporin. In fact, obtaining a key intermediate such as acetyl voclosporin in crystalline form allows for more efficient purification thereof, which benefits the entire process.

In a still further ancillary aspect, the present invention also relates to a process for obtaining an amorphous form of voclosporin of formula I

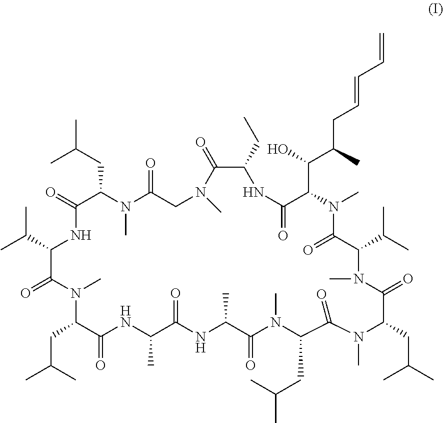 [SMILES: C=C/C=C/C[C@@H](C)[C@@H](O)[C@H]1C(=O)N[C@@H](CC)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

comprising the steps of:
   preparing a solution of voclosporin of formula I in a solvent/antisolvent system, wherein said solvent is selected from the group consisting of MTBE, THF, 2-methyl THF, diisopropyl ether, acetonitrile, acetone, ethanol and cyclopentylmethylether and said antisolvent is a hydrocarbon selected from the group consisting of normal heptane, cyclohexane and pentane; and
   recovering by precipitation from the solution thus prepared said voclosporin in amorphous form.

Indeed, the precipitation of voclosporin according to the invention allows to obtain an amorphous form of the product characterised by high stability over time and easy handling due to its powdery and non-sticky nature.

### BRIEF DESCRIPTION OF THE DRAWINGS

FIG. 1 shows the XRPD spectrum of the crystalline acetyl cyclosporine A of formula II obtained according to Example 2;

FIG. 2 shows the chromatogram obtained from example 3.

FIG. 3 shows the XRPD spectrum of the crystalline acetyl cyclosporine A of formula II obtained according to Example 3;

FIG. 4 shows the chromatogram obtained in Example 4 when the reaction is carried out in the presence of a base selected from dimethylpyridine (or lutidine) and 2,4,6-trimethylpyridine (or sym-collidine).

FIG. 5 shows the chromatogram obtained in Example 4 when the reaction is carried out in the absence of a base selected from dimethylpyridine (or lutidine) and 2,4,6-trimethylpyridine (or sym-collidine).

FIG. 6 shows the XRPD spectrum of crystalline acetyl voclosporin obtained according to Example 12 and 13;

FIG. 7 shows the XRPD spectrum of amorphous voclosporin obtained according to Example 17 and 18;

FIG. 8 shows the comparison of the XRPD spectra of the amorphous voclosporin samples at the end of the stability tests according to Example 19;

### DETAILED DESCRIPTION OF THE INVENTION

The present invention relates, in a first aspect thereof, to a process for the preparation of voclosporin of formula I in amorphous form

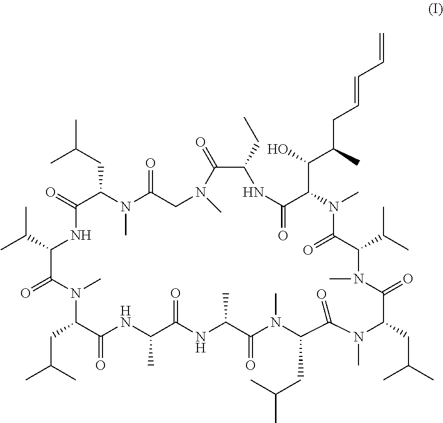 [SMILES: C=C/C=C/C[C@@H](C)[C@@H](O)[C@H]1C(=O)N[C@@H](CC)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

comprising the steps of:
a. acetylating cyclosporine A in the presence of methyl tert-butyl ether (MTBE) with acetic anhydride in a stoichiometric amount with respect to cyclosporine A, thus obtaining acetyl cyclosporine A of formula II:

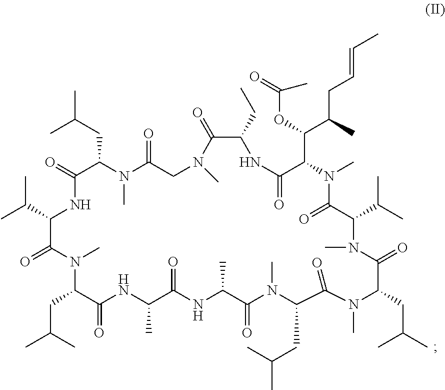 [SMILES: C/C=C/C[C@@H](C)[C@@H](OC(C)=O)[C@H]1C(=O)N[C@@H](CC)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

b. oxidising the acetyl cyclosporine A of formula II with at least one oxidising agent that is an osmiate salt in the presence of a base selected from the group consisting of dimethylpyridine (or lutidine) and 2,4,6-trimethylpyridine (or sym-collidine), wherein said base is in an amount such that a pH value in the range from 6 to 8 is achieved, and in the presence of a co-oxidant selected from the group consisting of a periodate of an alkali or alkaline earth metal and a hypochlorite of an alkali or alkaline earth metal, thus obtaining acetyl cyclosporine A aldehyde of formula III:

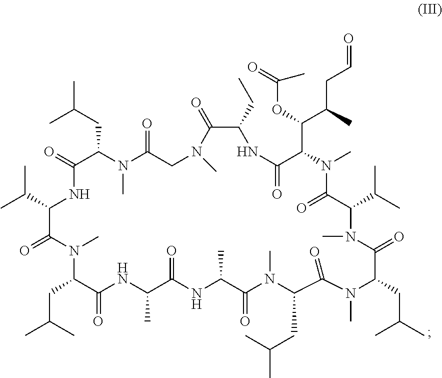 [SMILES: CC[C@@H]1NC(=O)[C@H]([C@H](OC(C)=O)[C@H](C)CC=O)N(C)C(=O)[C@H](C(C)C)N(C)C(=O)[C@H](CC(C)C)N(C)C(=O)[C@H](CC(C)C)N(C)C(=O)[C@@H](C)NC(=O)[C@H](C)NC(=O)[C@H](CC(C)C)N(C)C(=O)[C@H](C(C)C)NC(=O)[C@H](CC(C)C)N(C)C(=O)CN(C)C1=O]

c. converting the acetyl cyclosporine A aldehyde of formula III into acetyl voclosporin of formula IV:

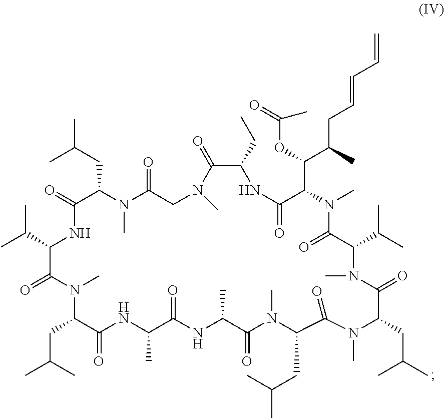 [SMILES: C=C/C=C/C[C@@H](C)[C@@H](OC(C)=O)[C@H]1C(=O)N[C@@H](CC)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

d. crystallising the acetyl voclosporin of formula IV with a solvent system selected from the group consisting of THF/n-heptane and MTBE, thus obtaining acetyl voclosporin in crystalline form;
e. hydrolysing the acetyl voclosporin of formula IV, thus obtaining voclosporin of formula I; and
f. isolating the voclosporin of formula I obtained in step e. obtaining said voclosporin of formula I in amorphous form, wherein said isolation is carried out by precipitation with a solvent/anti-solvent system, wherein said solvent is selected from the group consisting of MTBE, THF, 2-methyl THF, diisopropyl ether, acetonitrile, acetone, ethanol and cyclopentylmethylether, and said anti-solvent is a hydrocarbon selected from the group consisting of normal heptane, cyclohexane and pentane. In fact, it was surprisingly discovered that the use of MTBE with acetic anhydride in stoichiometric amounts in step a. made it possible to avoid large consumption of acetic anhydride, a toxic reagent with a high environmental impact, improving the reaction yield and purity with respect to the process applied in the prior art.

Furthermore, advantageously and surprisingly, by using an osmiate salt as the oxidising agent in step b. and a co-oxidant selected from the group consisting of a periodate of an alkali or alkaline earth metal and a hypochlorite of an alkali or alkaline earth metal, the oxidation reaction of acetyl cyclosporine A to obtain acetyl cyclosporine A aldehyde was particularly advantageous in terms of yield and conduction simplicity. The presence of the base selected from the group consisting of dimethylpyridine (or lutidine) and 2,4,6-trimethylpyridine (or sym-collidine) advantageously allowed the carboxylic acid impurity to be suppressed, as will become clear from the experimental part.

It should also be noted that, thanks to the use of a solvent system selected from the group consisting of THF/n-heptane and MTBE for the isolation of acetyl voclosporin, it was also advantageously possible to obtain this intermediate product in crystalline form. The crystalline acetyl voclosporin of the invention exhibits a X-ray powder diffraction spectrum (XRPD), with peaks at characteristic angle values 2ϑ(±0.2) of 5.0, 5.5, 7.0, 8.5, 8.8, 10.2, 12.4, 16.5, 17.2 and 20.9.

Furthermore, the Applicant has surprisingly discovered that by precipitating voclosporin with a solvent/antisolvent system, wherein said solvent is selected from the group consisting of MTBE, THF, 2-methyl THF, diisopropyl ether, acetonitrile, acetone, ethanol and cyclopentylmethylether, and said antisolvent is a hydrocarbon selected from the group consisting of normal heptane, cyclohexane and pentane, it is possible to obtain an amorphous form of the product characterised by high long-term stability and easy handling due to its powdery and non-sticky nature.

Within the context of the present description and following claims, all the numerical magnitudes indicating quantities, parameters, percentages, and so on are to be considered preceded in every circumstance by the term “about” unless indicated otherwise. Further, all the ranges of numerical magnitudes include all the possible combinations of maximum and minimum numerical values and all the possible intermediate ranges, as well as those indicated below.

In the present invention, when dimethylpyridine (or lutidine) is referred to, it means the compound 2,4-dimethylpyridine or the compound 2,6-dimethylpyridine, preferably 2,6-dimethylpyridine. The present invention can be presented in one or more of its aspects or one or more of the preferred characteristics reported below, which can be combined with one another according to the application requirements.

The process according to the present invention comprises the step a. of acetylating cyclosporine A in the presence of methyl tert butyl ether (MTBE) with acetic anhydride in a stoichiometric amount with respect to cyclosporine A, thus obtaining acetyl cyclosporine A of formula II:

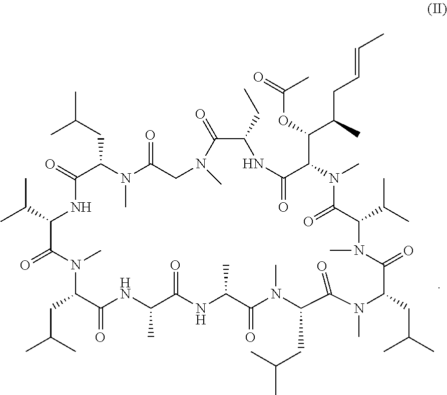 [SMILES: C/C=C/C[C@@H](C)[C@@H](OC(C)=O)[C@H]1C(=O)N[C@@H](CC)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

As advantageously indicated above, the use of MTBE and acetic anhydride in stoichiometric amounts made it possible to avoid large consumption of acetic anhydride. Indeed, acetic anhydride is known to be a toxic reagent with a high environmental impact. The use of stoichiometric amounts of acetic anhydride in the presence of MTBE was certainly surprising, not only because of its low environmental impact, but also because it allowed an improvement in reaction and purity with respect to the process applied in the prior art.

Said acetylation reaction is preferably conducted at a temperature in the range from 10 to 50° C., preferably 25 to 40° C. advantageously using one or more solvents and one or more reagents capable of forming an acetate group from a hydroxyl group. The acetylation of cyclosporine of step a. can be advantageously conducted with acetic anhydride and dimethylaminopyridine; acetic anhydride and pyridine; acetic anhydride, pyridine and dimethylaminopyridine; acetic anhydride and sodium acetate; acetic anhydride and p-toluenesulphonic acid; acetyl chloride, pyridine and dimethylaminopyridine; and a ketene.

At the end of the acetylation reaction, acetyl cyclosporine A is advantageously recovered from the reaction mixture, e.g. by filtration through a Buchner filter and advantageously purified by washing with a suitable solvent.

In an advantageous aspect, the acetyl cyclosporine A of formula II was obtained in crystalline form.

The invention thus provides a step a1. following phase a. which consists of crystallising the acetyl cyclosporine A from a solvent system selected from the group consisting of MTBE/n-heptane and MTBE/cyclohexane.

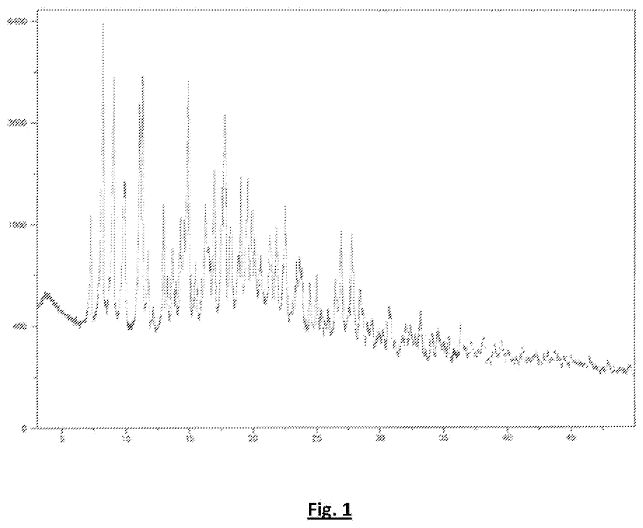

Acetyl cyclosporine A can be reproducibly obtained in crystalline form using the MTBE/n-heptane solvent system and has a X-ray powder diffraction spectrum (XRPD), with peaks at characteristic angle values 2ϑ (±0.2) of 8.3, 9.1, 11.1, 11.3, 13.0, 15.0, 17.0, 17.6, 17.8, 19.1 and 19.6. Therefore, the invention in a further aspect relates to a crystalline form of acetyl cyclosporine A of formula II having a X-ray powder diffraction spectrum (XRPD) with peaks at the characteristic angle values 2ϑ (±0.2) of 8.3, 9.1, 11.1, 11.3, 13.0, 15.0, 17.0, 17.6, 17.8, 19.1 and 19.6. The X-ray powder diffraction spectrum (XRPD) of acetyl cyclosporine A in crystalline form obtainable using the MTBE/n-heptane solvent system is shown in FIG. 1.

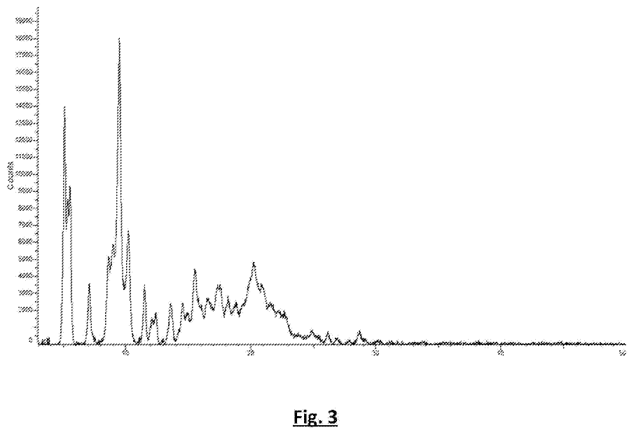

The acetyl cyclosporine A of formula II in crystalline form is also obtainable in reproducible form using the MTBE/cyclohexane solvent system and exhibits a X-ray powder diffraction spectrum (XRPD), with peaks at characteristic angle values 2ϑ (+0.2) of 5.1, 5.4, 5.5, 9.0, 9.5, 10.2, 15.5, 17.4, 20.2 and 20.9. The X-ray powder diffraction spectrum (XRPD) of acetyl cyclosporine A in crystalline form obtainable using the MTBE/cyclohexane solvent system is shown in FIG. 3.

The process according to the present invention comprises step b. of oxidising the acetyl cyclosporine A of formula II with at least one oxidising agent that is an osmiate salt in the presence of a base selected from the group consisting of dimethylpyridine (or lutidine) and 2,4,6-trimethylpyridine (or sym-collidine), wherein said base is in an amount such that a pH value in the range from 6 to 8 is achieved, and in the presence of a co-oxidant selected from the group consisting of a periodate of an alkali or alkaline earth metal and a hypochlorite of an alkali or alkaline earth metal, thus obtaining acetyl cyclosporine A aldehyde of formula III

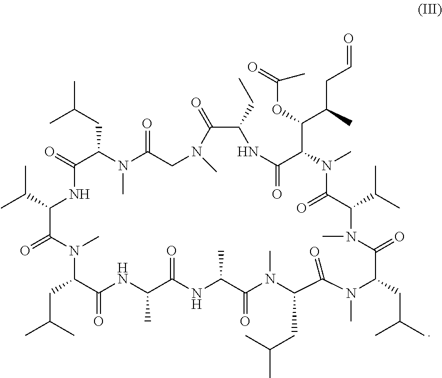 [SMILES: CC[C@@H]1NC(=O)[C@H]([C@H](OC(C)=O)[C@H](C)CC=O)N(C)C(=O)[C@H](C(C)C)N(C)C(=O)[C@H](CC(C)C)N(C)C(=O)[C@H](CC(C)C)N(C)C(=O)[C@@H](C)NC(=O)[C@H](C)NC(=O)[C@H](CC(C)C)N(C)C(=O)[C@H](C(C)C)NC(=O)[C@H](CC(C)C)N(C)C(=O)CN(C)C1=O]

As will be clear from the experimental part, in step b. the presence of the base selected from the group consisting of dimethylpyridine (or lutidine) and 2,4,6-trimethylpyridine (or sym-collidine) in such amount that a pH value in the range from 6 to 8 is achieved, preferably 6.5 to 7.5, advantageously allowed the suppression of the impurity carboxylic acid of formula

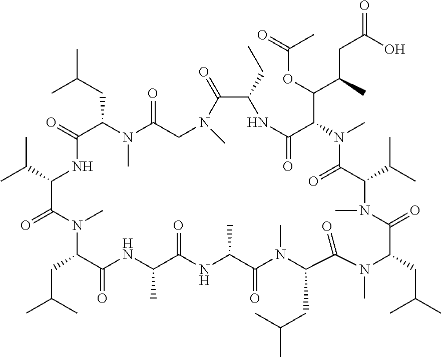 [SMILES: CC[C@@H]1NC(=O)[C@H](C(OC(C)=O)[C@H](C)CC(=O)O)N(C)C(=O)[C@H](C(C)C)N(C)C(=O)[C@H](CC(C)C)N(C)C(=O)[C@H](CC(C)C)N(C)C(=O)[C@@H](C)NC(=O)[C@H](C)NC(=O)[C@H](CC(C)C)N(C)C(=O)[C@H](C(C)C)NC(=O)[C@H](CC(C)C)N(C)C(=O)CN(C)C1=O]

Said base is preferably dimethylpyridine (or lutidine) in such amounts as to have a pH value in the range from 6 to 8, preferably 6.5 to 7.5.

The reaction of step b. of the process according to the invention is preferably conducted at a temperature in the range of 0-40° C., preferably 15-35° C.

The reaction is also preferably conducted in the presence of at least one suitable solvent or solvent mixture, preferably selected from the group consisting of: water and acetonitrile (CH₃CN).

Preferably, in said solvent mixture the ratio by volume H₂O:CH₃CN ranges from 1:1 to 1:5.

In a more preferred embodiment, said solvent mixture is an H₂O:CH₃CN mixture, preferably in a ratio by volume in the range from 1:1 to 1:3, more preferably about 1:1, 1:2 or 1:3, even more preferably about 1:3.

The at least one oxidising agent in step b. is an osmiate salt preferably used in amounts in the range from 0.01 to 0.1 equivalents per 1 equivalent of acetyl cyclosporine A; preferably 0.02 to 0.08 equivalents per 1 equivalent of acetyl cyclosporine A, e.g. in amounts of about 0.03 equivalents per 1 equivalent of acetyl cyclosporine A.

Preferably, said osmiate salt is an osmiate of an alkali or alkaline earth metal, more preferably selected from the group consisting of: potassium osmiate (K₂OsO₄) and sodium osmiate (Na₂OsO₄).

In a particularly preferred embodiment, the at least one oxidising agent is potassium osmiate (K₂OsO₄).

The reaction is conducted in the presence of at least one co-oxidant selected from the group consisting of a periodate of an alkali or alkaline earth metal and a hypochlorite of an alkali or alkaline earth metal. This alkali or alkaline earth metal is preferably selected from the group consisting of sodium and potassium.

In a particularly preferred embodiment, said at least one co-oxidant is sodium periodate (NaIO₄).

Preferably, said co-oxidant is used in amounts in the range from 1 to 5 equivalents per 1 equivalent of acetyl cyclosporin A; preferably 1.8 to 4.5 equivalents per 1 equivalent of acetyl cyclosporine A, e.g. in an amount of about 2.1 equivalents per 1 equivalent of acetyl cyclosporine A.

In a particularly preferred embodiment, this co-oxidant is sodium periodate, preferably used in amounts of about 2.1 equivalents per 1 equivalent of acetyl cyclosporine A.

The oxidising agent and/or co-oxidant may be added to the reaction mixture in any manner known to a person skilled in the art, e.g. they may be added at the start of the reaction in a single aliquot or, divided into several equal or different aliquots over a predetermined time.

Advantageously, at the end of the reaction, the acetyl cyclosporine A aldehyde of formula III is separated from the reaction mixture and purified. Said purification is advantageously carried out by separating the acetyl cyclosporine A aldehyde from the reaction mixture e.g. by filtration and then redissolving it with a suitable solvent, e.g. Me-THF.

The process according to the present invention thus comprises the step c. of converting the acetyl cyclosporine A aldehyde of formula III into acetyl voclosporin of formula IV:

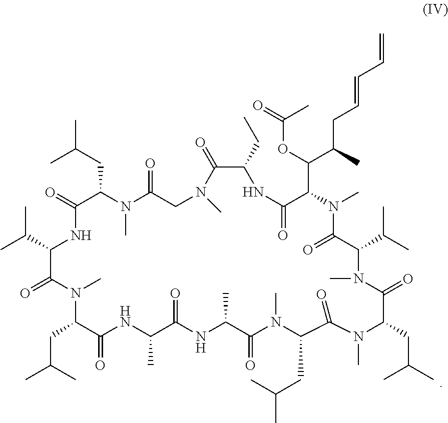 [SMILES: C=C/C=C/C[C@@H](C)C(OC(C)=O)[C@H]1C(=O)N[C@@H](CC)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

Preferably, the conversion reaction of acetyl cyclosporine A aldehyde of formula III to acetyl voclosporin of formula IV is advantageously conducted by means of the Peterson reaction (also known as Peterson olefination) or by means of the Wittig reaction.

Both the Peterson and the Wittig reaction are reactions well known to a person skilled in the art and commonly used in the field of organic synthesis.

In a preferred embodiment, step c. of the process is conducted by means of the Peterson reaction in the presence of at least one α-silyl-carbanion to provide a corresponding hydroxy-silane.

Preferably said α-silyl-carbanion is selected from the group consisting of: allyltrimethylsilane and the compound of formula V

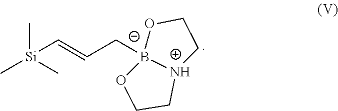

Preferably, said α-silyl-carbanion is used in amounts ranging from 1 to 1.5 equivalents per 1 equivalent of acetyl cyclosporine A aldehyde, more preferably from 1.1 to 1.4 equivalents per 1 equivalent of acetyl cyclosporine A aldehyde, even more preferably in amounts of about 1.2 equivalents per 1 equivalent of acetyl cyclosporine A aldehyde.

Preferably, in step c. the Peterson reaction takes place in the presence of an organo-lithium compound.

Preferably, said organo-lithium compound is an alkyl-lithium, preferably selected from the group consisting of: n-butyl lithium, s-butyl lithium, tert-butyl lithium, n-hexyl lithium, and mixtures thereof.

In a preferred embodiment of the invention, said organo-lithium compound is n-butyl lithium.

Said organo-lithium compound may be added to the mixture of step c. in any form known to a person skilled in the art, e.g. as a solution in a hydrocarbon solvent. In one embodiment, for example, said organo-lithium compound is added by means of a 15% by weight hexane solution of said organo-lithium compound.

Said organo-lithium compound may also be added to the mixture of step c, in any manner known to a person skilled in the art in a single aliquot or, divided into several equal or different aliquots, over a predetermined time.

Preferably, in step c. in said Peterson reaction the hydroxy silane formed by addition of said α-silyl-carbanion is hydrolysed under conditions of acid hydrolysis, preferably at pH comprised between 0 and 3, using an acid preferably selected from the group consisting of sulphuric acid (H₂SO₄) and hydrochloric acid (HCl).

Preferably, said step c. is conducted in the presence of a suitable solvent, more preferably selected from the group consisting of: dichloromethane (DCM), water, THF and mixtures thereof.

In one embodiment, said step c. is conducted in the presence of a solvent consisting of a DCM:water mixture, preferably in a 2:1 ratio by volume.

In a further embodiment, said step c. is conducted in the presence of THE as solvent or in the presence of a THF:water mixture, preferably in a 1:1 ratio by volume.

Advantageously, at the end of the reaction, acetyl voclosporin of formula IV is separated from the reaction mixture and purified. Such purification is advantageously carried out by separating the product from the reaction mixture, e.g. by filtration, and then re-mixing it with a suitable solvent.

Advantageously, the process according to the present invention thus comprises the optional step d. of crystallising the acetyl voclosporin of formula IV with a solvent system chosen from the group consisting of THF/n-heptane and MTBE, thereby obtaining acetyl voclosporin in crystalline form. Thus, in a preferred and advantageous aspect, due to the use of a solvent system selected from the group consisting of THF/n-heptane and MTBE for the separation of acetyl voclosporin, it was also advantageously possible to obtain said intermediate product in a crystalline form which is always reproducible.

Said crystalline form can be characterised by X-ray powder diffraction spectroscopy (XRPD) using CuKa radiation, with peaks at the characteristic angle values 2ϑ (±0.2) of 5.0, 5.5, 7.0, 8.5, 8.8, 10.2, 12.4, 16.5, 17.2 and 20.9.

Thus, in a further advantageous aspect, the invention concerns crystalline acetyl voclosporin having a X-ray powder diffraction spectrum (XRPD) with peaks at characteristic angle values 2ϑ (±0.2) of 5.0, 5.5, 7.0, 8.5, 8.8, 10.2, 12.4, 16.5, 17.2 and 20.9.

Preferably in an embodiment with the THF/n-heptane solvent system, the volume ratio THF to n-heptane ranges from 3:8 to 5:8, more preferably about 4:8.

Preferably in a further embodiment, with the MTBE solvent system, the volume used is in the range from 30 to 40 volumes, preferably around 35 volumes.

At the end of step d., acetyl voclosporin in crystalline form is obtained.

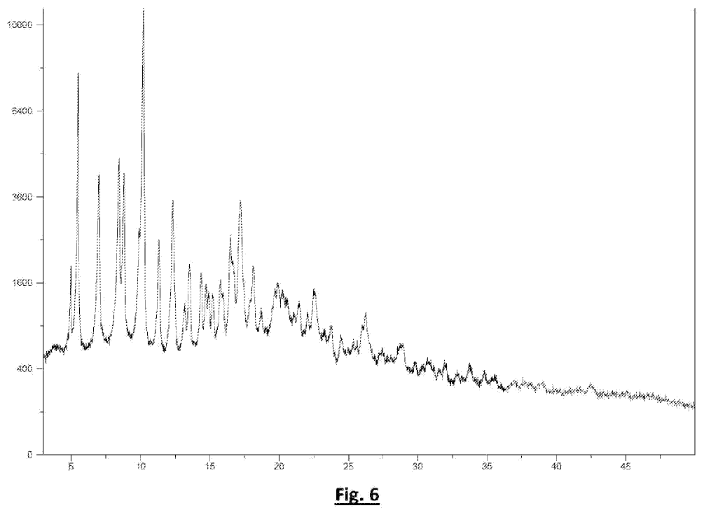

An X-ray powder diffraction spectrum (XRPD) of acetyl voclosporin in crystalline form obtained according to the present invention is shown by way of example in FIG. 6.

The process according to the present invention comprises the step e. of hydrolysing acetyl voclosporin of formula IV, thereby obtaining voclosporin.

The hydrolysis of acetyl voclosporin in step e. of the process according to the present invention can be conducted according to any of the methods known for the purpose to a person skilled in the art.

Said hydrolysis reaction is preferably conducted at a temperature in the range from 0 to 30° C., preferably 10 to 20° C. advantageously using one or more solvents and one or more bases capable of hydrolysing the acetate group of acetyl voclosporin.

The hydrolysis of the step e. can advantageously be conducted in a solvent selected from the group consisting of: methanol (MeOH), water, ethanol, isopropanol, normal-propanol and mixtures thereof; preferably it is conducted in methanol or with a methanol:water mixture in which the MeOH:water volume ratio is in the range from 10:1 to 1:1, more preferably it is about 3:1.

Preferably, at least one base in step e. is added, which can be any base known to a person skilled in the art for the hydrolysis of an acetate group.

Preferably, said base is selected from the group consisting of: a carbonate of an alkali or alkaline earth metal.

In an embodiment, said base is potassium carbonate (K₂CO₃).

The process according to the present invention thus comprises the step f. of isolating the voclosporin of formula I obtained in step e. obtaining said voclosporin of formula I in amorphous form, wherein said isolation is carried out by precipitation with a solvent/anti-solvent system, wherein said solvent is selected from the group consisting of MTBE, THF, 2-methyl THF, diisopropyl ether, acetonitrile, acetone, ethanol and cyclopentylmethylether, and said anti-solvent is a hydrocarbon selected from the group consisting of normal heptane, cyclohexane and pentane. Preferably, when said step f. involves the use of said solvent/anti-solvent system, the solvent:anti-solvent volume ratio is in the range from 1:1 to 1:15, more preferably 1:5 to 1:15, even more preferably 1:6 to 1:10.

In one embodiment, said step f. comprises the steps of (f-i-1) dissolving voclosporin in MTBE; (f-i-2) precipitating voclosporin from the solution at room temperature by removing MTBE.

In a further embodiment, said step f. comprises the steps of (f-ii-1) dissolving the voclosporin in a solvent, preferably MTBE; (f-ii-2) adding the antisolvent, preferably n-heptane, so as to have a solvent volume ratio, preferably MTBE, to the antisolvent in the range from 1:1 to 1:10, preferably about 1:6, until a suspension is formed; (f-ii-3) isolating voclosporin thus precipitated. Preferably, in said step (f-ii-2), said antisolvent is added in at least two equal or different aliquots at predetermined time intervals.

In a further embodiment, said step f. comprises the steps of (f-iii-1) dissolving voclosporin in a solvent, preferably MTBE; (f-iii-2) adding the antisolvent, preferably cyclohexane, so as to have a solvent volume ratio, preferably MTBE, to the antisolvent from 1:6 to 1:15, preferably about 1:10, until a suspension is formed; (f-iii-3) separating voclosporin thus precipitated.

In a still further embodiment, said step f. involves step f.iv-1 of dissolving voclosporin in a solvent, preferably MTBE, THF, acetonitrile, acetone, ethanol, resulting in a solution of voclosporin, and step f.iv-2 of dripping the solution of voclosporin obtained in step f.iv-1 into an antisolvent selected from the group consisting of normal-heptane, cyclohexane and pentane. Preferably in this further embodiment the solvent to antisolvent ratio is in the range from 1:6 to 1:15, more preferably 1:10.

At the end of step f., voclosporin is obtained in amorphous form.

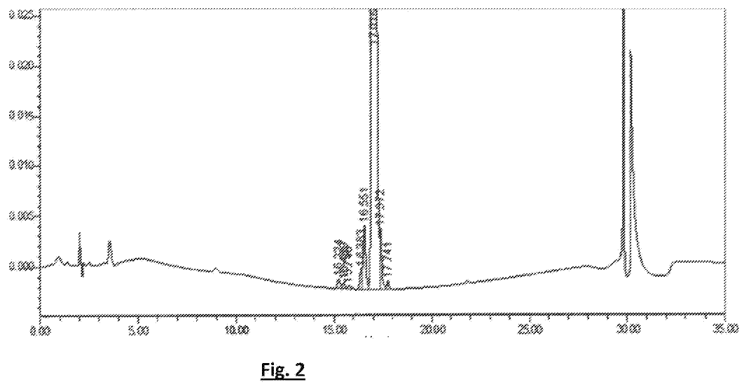

An X-ray powder diffraction spectrum (XRPD) of voclosporin in amorphous form obtained according to the present invention is shown by way of example in FIG. 2.

Depending on the various improvements made in the different synthesis steps starting from cyclosporine A and leading to the preparation of voclosporin, the present invention also has certain ancillary aspects.

In a further aspect thereof, the present invention relates to a process for the preparation of acetyl cyclosporine A aldehyde of formula III:

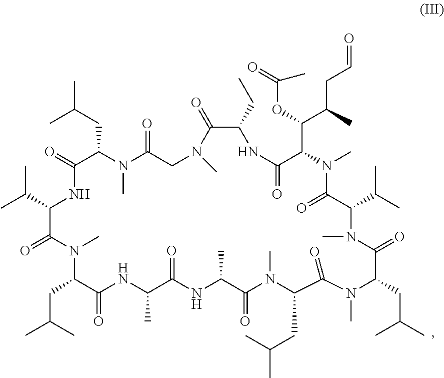 [SMILES: CC[C@@H]1NC(=O)[C@H]([C@H](OC(C)=O)[C@H](C)CC=O)N(C)C(=O)[C@H](C(C)C)N(C)C(=O)[C@H](CC(C)C)N(C)C(=O)[C@H](CC(C)C)N(C)C(=O)[C@@H](C)NC(=O)[C@H](C)NC(=O)[C@H](CC(C)C)N(C)C(=O)[C@H](C(C)C)NC(=O)[C@H](CC(C)C)N(C)C(=O)CN(C)C1=O]

comprising the steps of:
A. acetylating cyclosporine A in the presence of methyl tert-butyl ether (MTBE) with acetic anhydride in a stoichiometric amount with respect to cyclosporine A, thus obtaining acetyl cyclosporine A of formula II:

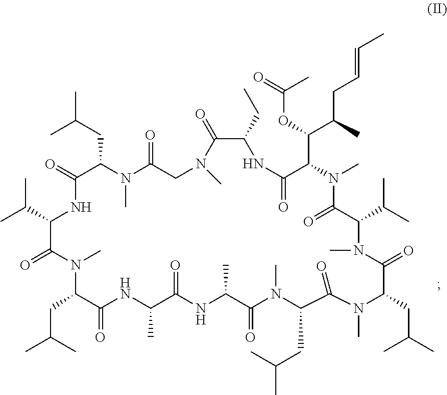 [SMILES: C/C=C/C[C@@H](C)[C@@H](OC(C)=O)[C@H]1C(=O)N[C@@H](CC)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

B. oxidising the acetyl cyclosporine A of formula II with at least one oxidising agent that is an osmiate salt in the presence of a base selected from the group consisting of dimethylpyridine (or lutidine) and 2,4,6-trimethylpyridine (or sym-collidine), wherein said base is in an amount such that a pH value in the range from 6 to 8 is achieved, and in the presence of a co-oxidant selected from the group consisting of a periodate of an alkali or alkaline earth metal and a hypochlorite of an alkali or alkaline earth metal, thus obtaining acetyl cyclosporine A aldehyde of formula III:

As advantageously indicated above, the use of MTBE and acetic anhydride in stoichiometric amounts made it possible to avoid large consumption of acetic anhydride. According to the invention, the use of stoichiometric amounts of acetic anhydride in the presence of MTBE not only allowed a low environmental impact, but an improvement in reaction and purity with respect to the process applied in the prior art.

The invention provides a step A1. subsequent to step A. which consists of crystallising acetyl cyclosporine A from a solvent system selected from the group consisting of MTBE/n-heptane and MTBE/cyclohexane.

At the end of step A1. acetyl cyclosporine A is in crystalline form. It can be reproducibly obtained in crystalline form through the use of the MTBE/n-heptane solvent system and exhibits a powder X-ray diffraction spectrum (XRPD), with peaks at characteristic angle values 2ϑ (±0.2) of 8.3, 9.1, 11.1, 11.3, 13.0, 15.0, 17.0, 17.6, 17.8, 19.1 and 19.6. Therefore, the invention in a further aspect relates to a crystalline form of acetyl cyclosporine A of formula II having a X-ray powder diffraction spectrum (XRPD) with peaks at the characteristic angle values 2ϑ (±0.2) of 8.3, 9.1, 11.1, 11.3, 13.0, 15.0, 17.0, 17.6, 17.8, 19.1 and 19.6. The X-ray powder diffraction spectrum (XRPD) of acetyl cyclosporine A in crystalline form obtainable using the MTBE/n-heptane solvent system is shown in FIG. 1.

The acetyl cyclosporine A of formula II in crystalline form is also obtainable in reproducible form using the MTBE/cyclohexane solvent system and exhibits an X-ray powder diffraction spectrum (XRPD), with peaks at characteristic angle values 2ϑ (+0.2) of 5.1, 5.4, 5.5, 9.0, 9.5, 10.2 and 15.56. The X-ray powder diffraction spectrum (XRPD) of acetyl cyclosporine A in crystalline form obtainable using the MTBE/cyclohexane solvent system is shown in FIG. 2.

The process according to the present invention comprises step B. of oxidising the acetyl cyclosporine A of formula II with at least one oxidising agent which is an osmiate salt in the presence of a base selected from the group consisting of dimethylpyridine (or lutidine) and 2,4,6-trimethylpyridine (or sym-collidine), thereby obtaining acetyl cyclosporine A aldehyde of formula III:

Other advantages and preferred conditions of conducting steps A., A1. and B. of this further aspect of the invention have already been described with reference to steps a., a1. and b. of the first aspect of the invention and are therefore not repeated herein.

In fact, as will be clear from the experimental part, in step B., corresponding to step b. of the first aspect, the presence of the base selected from the group consisting of dimethylpyridine (or lutidine) and 2,4,6-trimethylpyridine (or sym-collidine) advantageously allowed the suppression of the impurity carboxylic acid of formula

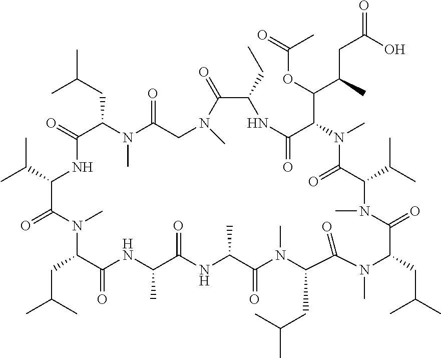 [SMILES: CC[C@@H]1NC(=O)[C@H](C(OC(C)=O)[C@H](C)CC(=O)O)N(C)C(=O)[C@H](C(C)C)N(C)C(=O)[C@H](CC(C)C)N(C)C(=O)[C@H](CC(C)C)N(C)C(=O)[C@@H](C)NC(=O)[C@H](C)NC(=O)[C@H](CC(C)C)N(C)C(=O)[C@H](C(C)C)NC(=O)[C@H](CC(C)C)N(C)C(=O)CN(C)C1=O]

The advantageousness in terms of yield and simplicity of handling the preparation of the intermediate product acetyl cyclosporine A aldehyde resulting from the conditions of steps A., B. and, preferably A., A1, and B. is indeed an advantage for the preparation of voclosporin, as it contributes substantially and even decisively to obtaining the desired product with high yield and under industrially scalable conditions. The Applicant has therefore found that this ancillary aspect of the invention included in its first and principal aspect independently represents a key innovative and advantageous aspect in the context of the processes for obtaining voclosporin.

In further aspects thereof, the present invention also relates to a process for obtaining acetyl voclosporin of formula IV in crystalline form and to the acetyl voclosporin in crystalline form thus obtained.

In particular, the present invention also relates to a process for obtaining acetyl voclosporin of formula IV in crystalline form

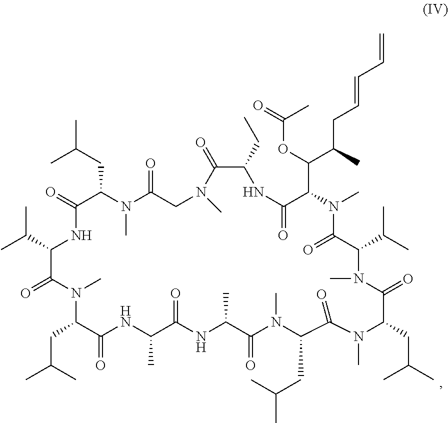 [SMILES: C=C/C=C/C[C@@H](C)C(OC(C)=O)[C@H]1C(=O)N[C@@H](CC)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

comprising the steps of:
   preparing acetyl voclosporin, for example but not exclusively according to steps a. to c. of the process according to the first aspect of the invention;
   crystallising acetyl voclosporin with a solvent system selected from the group consisting of THF/n-heptane and MTBE, thus obtaining acetyl voclosporin IV in crystalline form.

Furthermore, the present invention relates to the new crystalline form of acetyl voclosporin obtainable in this manner, namely to a crystalline form of acetyl voclosporin exhibiting an X-ray powder diffraction spectrum (XRPD) with peaks at the characteristic angle values 2ϑ (±0.2) of 5.0, 5.5, 7.0, 8.5, 8.8, 10.2, 12.4, 16.5, 17.2 and 20.9.

The possibility of obtaining acetyl voclosporin in crystalline form also represents an advantage within the process according to the first and main aspect of the invention but also, independently, a key innovative and advantageous aspect in the context of the processes for obtaining voclosporin. In fact, obtaining a key intermediate such as acetyl voclosporin in crystalline form allows for more efficient purification thereof, which benefits the entire process.

An X-ray powder diffraction spectrum (XRPD) of acetyl voclosporin in crystalline form obtained according to the present invention is shown by way of example in FIG. 6. Other advantages and preferred conditions of conducting the steps of this further aspect of the invention have already been described with reference to steps a. to c. and d, of the first aspect of the invention and are therefore not repeated here.

In yet another aspect thereof, the present invention relates to a process for obtaining an amorphous form of voclosporin of formula I:

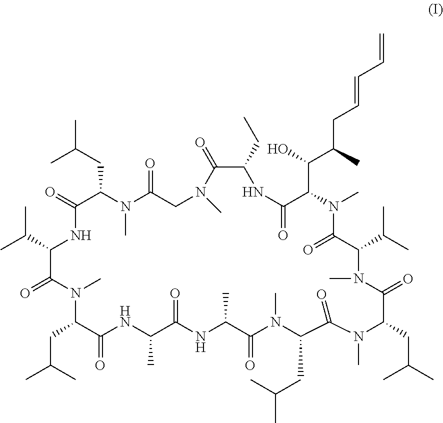 [SMILES: C=C/C=C/C[C@@H](C)[C@@H](O)[C@H]1C(=O)N[C@@H](CC)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]

comprising the steps of:
   preparing a solution of voclosporin in a solvent/antisolvent system, wherein said solvent is selected from the group consisting of MTBE, THF, 2-methyl THF, diisopropyl ether, acetonitrile, acetone, ethanol and cyclopentylmethylether and said antisolvent is a hydrocarbon selected from the group consisting of normal heptane, cyclohexane and pentane;
   recovering by precipitation from the solution thus prepared said voclosporin in amorphous form.

Indeed, the precipitation of voclosporin according to the invention makes it possible to obtain an amorphous form of the product characterised by high stability over time and easy handling due to its powdery and non-sticky nature.

Other advantages and preferred conditions for conducting this further aspect of the invention have already been described with reference to step f. of the first aspect of the invention and are therefore not repeated here.

The invention is now illustrated by means of some Examples to be understood for illustrative and non-limiting purposes.

### EXPERIMENTAL PART

### Methods

XRPD analysis: to perform the analysis, the sample was analysed by X-ray diffractometry using an X-ray diffractometer (operating at a voltage of 45 kV, current of 40 mA, scanning speed of 0.025710 degrees per second, CuKα source, ϑ angle range from 3.0° to) 49.992°.
HPLC analysis: to perform the analysis, the sample was dissolved in methanol using the following instrumentation and conditions:
Detector: UV 210 nm
Column: Zorbax SB-C18 250 mm×4.6 mm, 5 μm
Temperature: 65° C.
Sampler temperature: 15° C.
Mobile phase A:
0.1% Formic acid mixed with 55.2% Acetonitrile/42.3% water/2.5% MTBE
Mobile phase B: 0.1% Formic acid in Acetonitrile
Injection volume: 10 μL
Flow: 1.5 mL/min
Run time: 28 min
Elution gradient:

| Time (min) | Mobile phase A (%) | Mobile phase B (%) |
|---|---|---|
| 0-1 | 100 | 0 |
| 1-11 | 100 → 40 | 0 → 60 |
| 11-25 | 40 | 60 |
| 25-25.2 | 40 → 100 | 60 → 0 |
| 25.2-28 | 100 | 0 |

### EXAMPLES

### Example 1—Step a. and a1. (A and A1) of the Invention

In a three-necked round-bottomed flask, cyclosporine A, 20 g (16.63 mmol), was dissolved in MTBE 200 ml (10 V). The white suspension was left under agitation until a clear solution appeared. Acetic anhydride 10.386 g (99.78 mmol; 6 eq) were added drop by drop to the solution. 4-dimethylaminopyridine 4.63 g (33.26 mmol; 2 eq) were added in portions to the solution while maintaining 25±5° C. After 1 h, the product began to precipitate providing a white suspension. The suspension was left under agitation for 48 h. To quench the reaction, 50 mi (2.5 V) of water was added drop by drop. The temperature was maintained at 25±5° C. using an ice bath. A light two-phase solution was formed. The organic phase was separated and, after the addition of 50 mL of water (1 V), an aqueous solution of NaHCO3 (8% w/w) was added until a pH of 6-6.5 was obtained. The resulting biphasic solution was separated, and the organic phase washed with brine. The organic phase was distilled under vacuum until 7 volumes of residue were obtained. 100 mL of n-heptane (5 V) were added drop by drop to the shaken solution at 25±5° C. Precipitation occurred. The white suspension was left under agitation at 25±5° C. for 16H. The suspension was cooled to −15° C. for 2 h and then filtered. The solid was washed twice with 10 mL of 1:1 MTBE/n-heptane solution. The solid was dried overnight at 45° C. The acetyl cyclosporine solid was collected as a dry powder. 18.21 g (88% yield).

### Example 2—Step a. and a1. (A and A1) of the Invention

In a three-necked round-bottomed flask, cyclosporine A, 20 g (16.63 mmol), was dissolved in MTBE 200 ml (10 V). The white suspension was left under agitation until a clear solution appeared. Acetic anhydride 10.386 g (99.78 mmol; 6 eq) was added drop by drop to the solution. The MTBE solution was heated to 45±5° C. 4-dimethylaminopyridine 4.63 g (33.26 mmol; 2 eq) was added in portions to the solution while maintaining 45±5° C. The solution was left under agitation for 6 h. The reaction was cooled to 25±5° C. and quenched with 50 ml (2.5 V) of water added drop by drop. The temperature was maintained at 25±5° C. using an ice bath. A light two-phase solution was formed. The organic phase was separated and, after the addition of 50 ml of water (1 V), an aqueous solution of NaHCO3 (8% w/w) was added until a pH of 6-6.5 was obtained. The resulting two-phase solution was separated, and the organic phase washed with brine. The organic phase was distilled under vacuum until 7 volumes of residue were obtained. 100 ml of n-heptane (5 V) were added drop by drop to the shaken solution at 25±5° C. Precipitation occurred. The white suspension was left under agitation at 25±5° C. for 16 h. The suspension was cooled to −15° C. for 2 h and then filtered. The solid was washed twice with 10 mL of 1:1 MTBE/n-heptane solution. The solid was dried overnight at 45° C. The acetyl cyclosporine solid was collected as a dry powder. 17.93 g (87% yield).

The solid obtained in example 1 and example 2 was thus analysed to obtain the X-ray powder diffraction spectrum (XRPD) of acetyl cyclosporine A in crystalline form shown in FIG. 1. The characteristic peaks of said crystalline form of angle 2ϑ(±0.2) were 8.3, 9.1, 11.1, 11.3, 13.0, 15.0, 17.0, 17.6, 17.8, 19.1 and 19.6.

### Example 3—Step a. and a1. (A and A1) of the Invention

2.0 kg of cyclosporine A (1.66 mol, 1.0 equiv.) were dissolved in 20 L of MTBE. Under agitation, 1.02 kg of acetic anhydride (9.96 mol, 6.0 equiv.) and 415.4 g of DMAP (3.32 mol, 2.0 equiv.) were added. The final mixture was heated to 45° C. and kept under agitation for 16 hours. The reaction mixture was cooled to 20° C. and quenched with 5.0 kg of water. The two phases were separated, and the organic phase was washed twice with 1M HCl (4.0 L×2) and once with 8% NaHCO₃ aqueous mixture: 15% brine (2.0 L+3.0 L). The final organic layer was concentrated to 8.0 L under reduced pressure and heated to 55° C. 10.0 L of cyclohexane were added and the suspension was cooled to 0° C. The suspension was filtered and the solid was washed once with MTBE mixture: Cyclohexane (0.8 L+1.0 L). The solid was dried at 50° C. under vacuum for 16 hours providing a white solid (1.86 kg, yield=90.0%, typical A % HPLC purity ≥98%).

FIG. 2 shows the chromatogram obtained from the reaction of step a. (A.), which demonstrates the high purity of crystalline acetyl cyclosporine A obtainable by using stoichiometric amounts of acetic anhydride in the presence of MTBE. The results are shown in the table below:

| | Retention | |
|---|---|---|
| | time | Area % |
| 1 | 15.224 | 0.09 |
| 2 | 15.489 | 0.05 |
| 3 | 15.746 | 0.03 |
| 4 | 16.383 | 0.14 |
| 5 | 16.551 | 0.56 |
| 6 | 17.016 | 98.68 |
| 7 | 17.372 | 0.39 |
| 8 | 17.741 | 0.06 |

The solid obtained in Example 3 was thus analysed to obtain the X-ray powder diffraction spectrum (XRPD) of acetyl cyclosporine A in crystalline form shown in FIG. 3. The characteristic peaks of the said crystalline form of angle 2ϑ(±0.2) were 5.1, 5.4, 5.5, 9.0, 9.5, 10.2, 15.5, 17.4, 20.2 and 20.9.

### Example 4—Step b. or B. Of the Process

Acetyl cyclosporine A 11.2 g (9.00 mmol; 1.0 eq) from example 1 was dissolved in 168 ml CH₃CN (15 V) to provide a clear solution. The solution was concentrated under vacuum to 8 volumes and CH₃CN was added at 15V. 56 mL of water (5 V) were added. To the resulting solution, 1.9 g of 2,6-lutidine (18 mmol; 2 eq) were added and the solution reached pH 7. The suspension was agitated at 25±5° C. for 30 minutes. Potassium osmate 0.050 g (0.136 mmol; 0.015 eq) was added in one portion. The solution appears brownish after dissolution of the osmate. After 15 minutes, 3.850 g NaIO₄ (18 mmol; 2 eq.) were added in five portions (0.770 g; 3.6 mmol; 0.4 eq. each) at a time interval of 1 hr apart. During the addition, a brown suspension was formed, and the resulting salts were dissolved by adding 50 ml of water. Then 62 mL of water were added, reaching 15V. The suspension was left under agitation for 16 h. 44.8 mL of IPAC (4V) were added followed by 10 ml of water, and the resulting two-phase solution was left under agitation for 30 minutes. The organic phase was separated, and the aqueous phase was extracted twice with 56 mL of IPAC (5V). Oxidants in each organic and aqueous phase were monitored with Quantofix Peroxide 100®, which shows no presence of oxidants. The organic phases were combined and concentrated at 5V. The concentrated organic phase was washed with 56 mL (5V) brine. The organic phase was concentrated and solvent-switched in THF (5 volumes of final solution). 200 mL of n-heptane (4V based on total residue) were added drop by drop in 1.5 hours. The resulting mixture was agitated at room temperature for 16 hours. Filtration yielded a white solid that was dried overnight at 40° C. 10.4 g of acetyl cyclosporin A aldehyde with a yield of 94% were obtained.

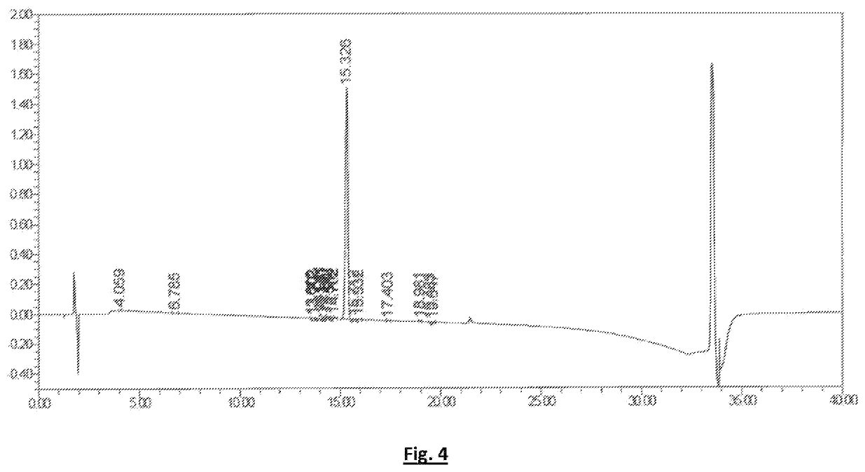

The solid was then subjected to HPLC analysis, which graph is shown in FIG. 4 and the results in the table below:

| | | Retention | | | Relative |
|---|---|---|---|---|---|
| | Name of Peak | time | Area | Area % | retention time |
| 1 | | 4.059 | 33219 | 0.23 | 0.26 |
| 2 | | 6.785 | 30756 | 0.21 | 0.44 |
| 3 | | 13.602 | 8703 | 0.06 | 0.89 |
| 4 | | 13.703 | 32306 | 0.23 | 0.89 |
| 5 | Acetyl cyclosporine A + 15 imp. | 14.011 | 193979 | 1.35 | |
| 6 | Acetyl cyclosporine A diol 1 | 14.141 | 52127 | 0.36 | |
| 7 | Acetyl cyclosporine A diol 2 | 14.301 | 105713 | 0.74 | |
| 8 | | 14.514 | 60990 | 0.43 | 0.95 |
| 9 | | 14.702 | 89670 | 0.63 | 0.96 |
| 10 | Acetyl cyclosporine A aldehyde | 15.326 | 13532983 | 94.49 | |
| 11 | | 15.712 | 60564 | 0.42 | 1.03 |
| 12 | | 15.932 | 33398 | 0.23 | 1.04 |
| 13 | Acetyl cyclosporine A | 17.403 | 15430 | 0.11 | |
| 14 | | 18.981 | 35967 | 0.25 | 1.24 |
| 15 | | 19.483 | 12927 | 0.09 | 1.27 |
| 16 | | 19.647 | 22894 | 0.16 | 1.28 |

As can be seen from the table, the impurities were very low and the carboxylic acid impurity (+16 imp.) amounted to only 1.35%. In order to assess the cruciality of the presence of a base in step b. (or B.), step b. (or B.) was repeated without the presence of the base.

Acetyl cyclosporine A 5.580 g (4.48 mmol; 1.0 eq) was dissolved in CH₃CN 55 ml (30 V) to provide a clear solution. 55 mL of water (10 V) were added. The suspension was agitated at 25±5° C. for 30 minutes. Potassium osmate 0.050 g (0.136 mmol; 0.03 eq) was added in one portion. The resulting suspension appears brownish after dissolution of the osmate. After 15 minutes, 2.012 g NaIO4 (9.408 mmol; 2.1 eq.) were added in three portions (0.670 g; 3.136 mmol; 0.7 eq. each) at an interval of 30 minutes apart. A brown suspension forms during addition. The suspension was left under agitation for 16 h at 25±5° C. 10 ml of a saturated solution of Na₂S₂O₃ 7.083 g (44.8 mmol; 10.0 eq) were added drop by drop to the reaction mixture. After 30 minutes under agitation, the oxidants were monitored with Quantofix Peroxide 100®. 5 ml of water (1 V) were added to the reaction mixture until the salts were dissolved and a homogeneous clear solution appeared. The solution was extracted three times with 100 ml Me-THF (20 V). The organic phases were collected and dried under vacuum. The result is a white solid, 5.33 g, with a yield of 96%.

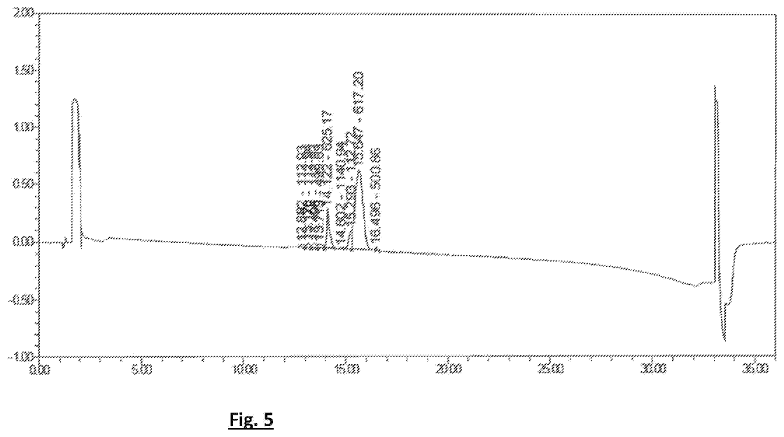

The product obtained was subjected to HPLC analysis as mentioned above and the graph shown in FIG. 5 and the results in the table below were obtained:

| | Name of | Retention | | | Relative |
|---|---|---|---|---|---|
| | Peak | time | Area | Area % | retention time |
| 1 | | 12.882 | 298343 | 1.18 | 0.82 |
| 2 | | 13.170 | 394675 | 1.56 | 0.84 |
| 3 | | 13.496 | 538772 | 2.13 | 0.86 |
| 4 | | 13.713 | 544396 | 2.16 | 0.88 |
| 5 | Ac_CsA_+16 | 14.122 | 3170032 | 12.56 | 0.90 |
| 6 | | 14.802 | 39261 | 0.16 | 0.95 |
| 7 | | 15.293 | 1438258 | 5.70 | 0.98 |
| 8 | Ac_CsA_CHO | 15.647 | 18651359 | 73.90 | |
| 9 | | 16.496 | 162004 | 0.64 | 1.05 |

As can be seen from the table above, the spectrum indicated the presence of high amounts of the impurity carboxylic acid (at 12.56%) of formula:

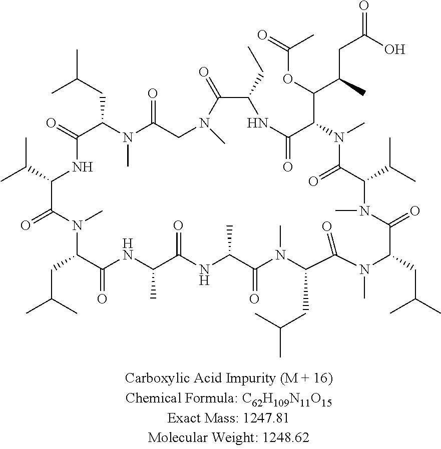 [SMILES: CC[C@@H]1NC(=O)[C@H](C(OC(C)=O)[C@H](C)CC(=O)O)N(C)C(=O)[C@H](C(C)C)N(C)C(=O)[C@H](CC(C)C)N(C)C(=O)[C@H](CC(C)C)N(C)C(=O)[C@@H](C)NC(=O)[C@H](C)NC(=O)[C@H](CC(C)C)N(C)C(=O)[C@H](C(C)C)NC(=O)[C@H](CC(C)C)N(C)C(=O)CN(C)C1=O]

The presence of the base chosen from the group consisting of dimethylpyridine (or lutidine) and 2,4,6-trimethylpyridine (or sym-collidine) in such amounts as to have a pH value in the range from 6 to 8 when performing step b. (or B.) was therefore extremely advantageous as it allowed for a drastic reduction in undesirable carboxylic acid impurity.

### Example 5—Preparation of α-Silyl-Carbanion of Formula V

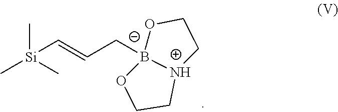

2.0 g (2.78 ml, 17.53 mmol) of allyl trimethylsilane were placed in a nitrogen atmosphere into a three-necked round-bottomed flask. 6 ml of dry THF (3 vol.) were added and the formation of a clear solution was observed. 11.5 ml of 1.6 M n-butyl lithium in hexane (18.40 mmol 1.05 eq) were then added drop by drop at 20° C. over 30 minutes until the solution turned yellow-orange; an ice bath was used to check the exothermy of the addition. The solution was left under agitation for 1 hour. The solution was then cooled to −78° C. with a dry ice bath in acetone. 3.460 g (4.24 ml 18.40 mmol 1.05 eq) of isopropyl borate were then added drop by drop. The reaction mixture was left under agitation for 1 hour until the colour turned from yellow to white. The reaction mixture was then poured into a 1M HCl solution (10 ml) until a pH value of 4-5 was obtained. The aqueous phase was discarded, and the organic phase collected. A solution of diethanolamine (1.750 g, 16.65 mmol 0.95 eq) in isopropylalcohol (iPrOH, 4 ml) was prepared separately and added drop by drop to the organic phase containing the boronic acid at 20° C. The resulting solution was left under agitation for at least 1 hour. 30 ml of n-heptane (15 vol.) were then added to provide a two-phase emulsion. The emulsion was concentrated until a white solid began to precipitate. 30 ml of n-heptane were added and concentrated to initial volume to remove traces of THF. The suspension was then filtered to obtain a white solid. The solid contained traces of diethanolamine so it was suspended in THF (4.4 vol, 8.8 ml) and filtered to obtain a white solid.

### Example 6—Preparation of α-Silyl-Carbanion of Formula V

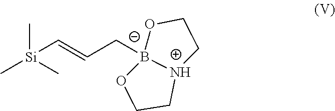

2.0 g (2.78 ml, 17.53 mmol) of allyl trimethylsilane were placed in a nitrogen atmosphere in a three-necked round-bottomed flask. 6 ml of dry THF (3 vol.) were added and the formation of a clear solution was observed. Then 11.5 ml of 1.6 M n-butyl lithium in hexane (18.40 mmol 1.05 eq) were added drop by drop at 20° C. over 30 minutes until the solution turned yellow-orange; an ice bath was used to check the exothermy of the addition. The solution was left under agitation for 1 hour. The solution was then cooled to −30° C. with a dry ice bath in acetone. 3.460 g (4.24 ml 18.40 mmol 1.05 eq) isopropyl borate were added drop by drop. The reaction mixture was left under agitation for 1 hour until the colour turned from yellow to white. The reaction mixture was then poured into a 1M HCl solution (10 ml) until a pH value of 4-5 was obtained. The aqueous phase was discarded, and the organic phase collected. A solution of diethanolamine (1.750 g, 16.65 mmol 0.95 eq) in isopropylalcohol (4 ml) was prepared separately and added drop by drop to the organic phase containing boronic acid at 20° C. The solution was left under agitation for 1 hour. 30 ml of n-heptane (15 vol.) were added until a two-phase emulsion appeared. The emulsion was concentrated until a white precipitate appeared. 30 ml of n-heptane were added and concentrated to the initial volume to remove traces of THF. The suspension was then filtered to obtain a white solid. The solid still contained traces of diethanolamine so it was suspended in THF (4.4 vol.) (8.8 ml) and filtered to obtain a white solid.

### Example 7—Process Step c

7.0 g (5.679 mmol) of acetyl cyclosporine A aldehyde obtained according to Example 4 (in the presence of the base 2,6-lutidine) and 1.740 g (7.659 mmol 1.2 eq. 90% purity) of the compound of formula V obtained according to Example 5 or 6 were dissolved in 63 ml of a DCM: H₂O 2:1 v/v (9 vol.) mixture resulting in a heterogeneous two-phase mixture. 1.022 mg (0.973 ml, 17.037 mmol, 3 eq) of concentrated acetic acid was added drop by drop to the reaction mixture in 30 minutes. The pH was monitored in this step, revealing a value of about 4.5. The reaction mixture was left under agitation overnight.

The organic phase was then separated, and the aqueous phase was extracted three times with 20 ml of dichloromethane (3 vol.). The organic extracts were pooled, and the solvent was changed to THF (40 ml 6 vol.), i.e. it was evaporated to dryness of the crude extract, which was then subjected to vacuum dissolution-concentration cycles in THF alone in order to remove any residual dichloromethane.

The reaction mixture was then cooled to 0° C. and 1.113 g (0.562 ml, 11.358 mmol 2 eq) of concentrated sulphuric acid were added drop by drop. The temperature was raised to 20° C. The pH was monitored and maintained at a value of about 1.5. The reaction mixture was left under agitation overnight at 20° C. A further 1.113 g (0.562 ml, 2 eq) of concentrated sulphuric acid were added drop by drop at 0° C. After completion of the reaction, 20 millilitres (3 vol.) of water were added drop by drop to the reaction mixture. A 2M NaOH solution was then added drop by drop to the reaction mixture until a pH of about 4.5 was reached. The solution was extracted three times with 35 ml MTBE (5 vol.) aliquots. The combined organic phases were concentrated at reduced pressure. The resulting white solid was dissolved in 100 ml MTBE (14 vol.) until crystallisation began. The suspension was left under agitation overnight at 20° C. Acetyl voclosporin (white solid, 5.35 g, 75% yield) was then obtained by filtration.

### Example 8—Process Step c

7.0 g (5.679 mmol) acetylcyclosporin A aldehyde obtained according to Example 4 (in the presence of the base 2,6-lutidine) and 1.740 g (7.659 mmol 1.2 eq. 90% purity) of the compound of formula V obtained according to Example 5 or 6 were dissolved in 63 ml (9 vol.) of DCM: H₂O 2:1 v/v obtaining a heterogeneous two-phase mixture. 1.022 mg/0.973 ml (17.037 mmol 3 eq) of concentrated acetic acid was added drop by drop to the reaction mixture in 30 minutes. The pH was checked and found to be about 4.5. The reaction mixture was left under agitation overnight. The organic phase was separated, and the aqueous phase was extracted three times with 20 ml aliquots (3 vol.) of dichloromethane. The organic phases were pooled, and the solvent was changed to THF (40 ml 6 vol.), i.e. it was evaporated to dryness of the crude extract, which was then subjected to vacuum dissolution-concentration cycles in THF alone in order to remove any residual dichloromethane.

The reaction mixture was cooled to 0° C. and 1.119 g/0.940 ml (11.358 mmol 2 eq) of concentrated HCl (37% HCl solution) was added drop by drop. The temperature was raised to 20° C. The pH was checked, which revealed a value of about 1.5. The reaction mixture was left under agitation overnight at room temperature. A further 1.119 g/0.940 ml (2 eq) of concentrated HCl (37% HCl solution) were added drop by drop at 0° C. After completion of the reaction 20 millilitres (3 vol.) of water were added drop by drop to the reaction mixture. A 2M NaOH solution was then added drop by drop to the reaction mixture until a pH of about 4.5 was reached. The solution was extracted three times with 35 ml aliquots of MTBE (5 vol.). The combined organic phases were concentrated at reduced pressure. The resulting white solid was suspended in 100 ml (14 vol.) of MTBE, resulting in crystallisation. The suspension was left under agitation overnight at room temperature and was then filtered, yielding a white solid (Acetyl Voclosporin, 5.35 g, 75% yield).

### Example 9—Process Step c

0.1 g (0.081 mmol) of acetylcyclosporin A aldehyde obtained according to Example 4 (in the presence of the base 2,6-lutidine) and 0.025 g (0.097 mmol 1.2 eq. 90% purity) of the compound of formula V obtained according to Example 5 or 6 were dissolved in THF (0.6 ml, 6 vol.). 0.015 mg/0.014 ml (0.243 mmol 3 eq) of concentrated acetic acid were added drop by drop to the reaction mixture in 30 minutes. The pH was checked and found to be about 4.5. 0.6 ml of H₂O (6 vol.) was then added to the reaction mixture to produce a two-phase mixture (THF: H₂O 1:1 v/v). The reaction mixture was left under agitation overnight. Additional 0.015 mg/0.014 ml (0.243 mmol 3 eq) of acetic acid were then added drop by drop. The organic phase was separated, and the aqueous phase was extracted three times with 3 ml (30 vol.) aliquots of DCM. The organic phases were combined, and the solvent was made up to THF (0.6 ml 6 vol.).

The reaction mixture was cooled to 0° C. and 0.016 g/0.009 ml (0.162 mmol 2 eq) of concentrated sulphuric acid were added drop by drop. The temperature was raised to 20° C. The pH was checked, which revealed a value of about 1.5. The reaction mixture was left under agitation overnight at 20° C. A further 0.016 g/0.009 ml (0.162 mmol 2 eq) of H₂SO₄ conc. 98% were added drop by drop at 0° C. After completion of the reaction, 1 millilitre (10 vol.) of water was then added drop by drop to the reaction mixture. A 2M NaOH solution was also added drop by drop to the reaction mixture until a pH of about 4.5 was reached. The solution was extracted three times with 5 ml aliquots of MTBE (50 vol.). The combined organic phases were concentrated under reduced pressure to produce a white solid (acetyl voclosporin, 88.16 mg, 88.3% yield).

### Example 10—Process Step c

1.0 g, (8.752 mmol) allyl trimethylsilane were placed in a nitrogen atmosphere in a three-necked round-bottomed flask. 7.4 ml (7.4 vol.) of THF were added and a clear solution was observed to form. 1.6 ml n-butyl lithium in 5.5 ml hexane (8.8 mmol 1.05 eq) were added drop by drop over 30 minutes until the solution turned from orange to yellow. The solution was then cooled to −78° C. with a dry ice bath in acetone. 1.646 g isopropylborate (2.02 ml 8.752 mmol 1 eq) were added. The reaction mixture was left under agitation for 2 hours until the colour changed from yellow to white. The reaction mixture was then poured into a 1M HCl solution (8 ml) until a pH value of about 4 was obtained. The aqueous phase was then extracted three times with 10 ml DCM. The DCM solution was concentrated until the boronic acid solution reached 10 wt %.

In a separate flask, 0.719 g (0.583 mmol 1 eq) of acetylcyclosporin A aldehyde obtained according to example 4 were dissolved in 4 ml DCM (5 vol.) and were then added to the previously prepared trimethylsilane allylboronic acid solution. The reaction mixture was left overnight at 20° C. Then 10 ml of THF were added and the solution was brought to 0° C. At this point, 228 mg (0.125 ml, 2 eq) of H₂SO₄ conc. 98% were added drop by drop and then the temperature was adjusted to about 20° C. A further 228 mg (0.125 ml, 2 eq) of H₂SO₄ conc. 98% were added drop by drop at 0° C. When the reaction was complete, a 2M NaOH solution was added drop by drop to the reaction mixture until a pH of about 4.5 was reached. The solution was extracted three times with 35 ml aliquots of MTBE (5 vol.). The combined organic phases were concentrated at reduced pressure. The product was obtained as a white solid (acetyl voclosporin, 685 mg, yield 93.5%).

### Example 11—Process Step d. Purification in THF/n-Heptane

Acetyl Voclosporin 127.0 g (0.101 mol: 1.0 eq) was dissolved in 500 mL (4V) THF. 1 L (8V) of n-heptane was added to the clear solution in 1 h 30 min. The resulting mixture was stirred for 16 hours at room temperature. The reaction mixture was cooled to 0-5° C. for 2 hours, then filtered to 110 g of the desired product. The resulting solid was dried at 40° C. under vacuum for 16 hours to obtain 100 g with a yield of 80%.

The resulting solid was subjected to analysis, obtaining the X-ray powder diffraction spectrum (XRPD) depicted in FIG. 6. The characteristic peaks of said crystalline form of angle 2ϑ(±0.2) were 5.0, 5.5, 7.0, 8.5, 8.8, 10.2, 12.4, 16.5, 17.2, 20.9.

### Example 12—Process Step d. Purification of Acetyl Voclosporin in MTBE

Acetyl Voclosporin 0.5 g was suspended in 17.5 mL (35 V) MTBE. The resulting mixture was heated until complete dissolution, then cooled to room temperature. The solid obtained was collected by filtration and dried at 40° C. under vacuum for 48 hours.

The resulting solid was subjected to analysis, obtaining the X-ray powder diffraction spectrum (XRPD) depicted in FIG. 6. The characteristic peaks of said crystalline form of angle 2ϑ(±0.2) were 5.0, 5.5, 7.0, 8.5, 8.8, 10.2, 12.4, 16.5, 17.2, 20.9.

The crystalline form of acetyl voclosporin was thus reproducible and obtainable with a solvent system selected from the group consisting of THF/n-heptane and MTBE.

### Example 13—Process Step e

2 g (1.590 mmoles) of acetyl Voclosporin obtained according to Example 11 were dissolved in 40 ml methanol (20 vol.). 1.099 g (7.95 mmol 5 eq) of potassium carbonate were added at 20° C., leading to the formation of a white suspension. After agitation for 5 hours at 20° C., the reaction mixture was diluted with 15 ml (7.5 vol.) of water until a clear solution was obtained (added drop by drop over 30 minutes while keeping the temperature below 25° C.). Most of the methanol was evaporated under reduced pressure until an aqueous suspension was formed and then 30 ml (15 vol.) of MTBE was added under agitation. The aqueous layer was separated and extracted with 2×30 ml (15 vol.) of MTBE. The combined organic phases were washed with brine and dried under reduced pressure at 45° C. A white solid was obtained (voclosporin, 1.91 g, yield 98.9%).

### Example 14—Process Step e

0.100 g (0.08 mmol) of acetyl Voclosporin obtained according to Example 11 were dissolved in 10.6 ml methanol (106 vol.). 0.264 g (1.911 mmol 24 eq) of potassium carbonate was added at 20° C., leading to the formation of a white suspension. After agitation overnight at room temperature, a clear solution was formed. The solution was then diluted with 1 ml (10 vol.) of water (H₂O). Most of the methanol was evaporated under reduced pressure until an aqueous suspension was formed and then 1.5 ml (15 vol.) of MTBE were added under agitation. The aqueous layer was separated and extracted with 2×1.5 ml (15 vol.) of MTBE. The combined organic phases were washed with brine and dried under reduced pressure at 45° C. A white solid was found to form (voclosporin, 92.6 mg, yield 95.3%).

### Example 15—Process Step e

500 mg (0.398 mmol) acetyl Voclosporin obtained according to Example 11 were dissolved in 53 ml methanol (106 vol.). 1.321 g (9.55 mmoles) of potassium carbonate (24 eq.) were added to the reaction mixture at 20° C. to form a white suspension. The suspension was left overnight at 20° C. Water was then added until the salts were completely dissolved. The solution was concentrated with the rotavapor until the precipitation of a solid was observed. The suspension was extracted three times with MTBE (20 ml, 40 vol.). The collected organic phases were concentrated under reduced pressure, leading to the formation of solid voclosporin.

### Example 16—Process Step e

0.250 g (0.199 mmoles) of acetyl voclosporin obtained according to Example 11 were dissolved in 5 ml of a MeOH:water 3:1 v/v (20 vol.) mixture. 0.138 g (0.995 mmol 5 eq) of potassium carbonate were added at 20° C. After agitation for 30 hours at room temperature, the mixture was concentrated under reduced pressure to evaporate most of the methanol until an aqueous suspension was formed. The residue was reconstituted in 3.75 ml (15 vol.) of MTBE. The aqueous layer was separated and extracted with MTBE 2×3.75 ml (15 vol.). The combined organic phases were washed with brine and dried under reduced pressure at 45° C. A white solid was obtained (voclosporin, 194.34 mg, yield 80.7%).

### Example 17—Process Step f

250 mg (206 mmol) of solid voclosporin obtained according to example 11 were dissolved in 1 ml MTBE (4 Vol.). 1.0 ml of n-heptane (1.2 ml) was added to form a white precipitate. The suspension was left under agitation for 10 minutes until the solution became clear. 1.4 ml of n-heptane were then added to form a turbid suspension and a further 3.3 ml of n-heptane were added, leaving the suspension under agitation. The suspension was filtered on a Buchner funnel to produce a solid (90.9 mg).

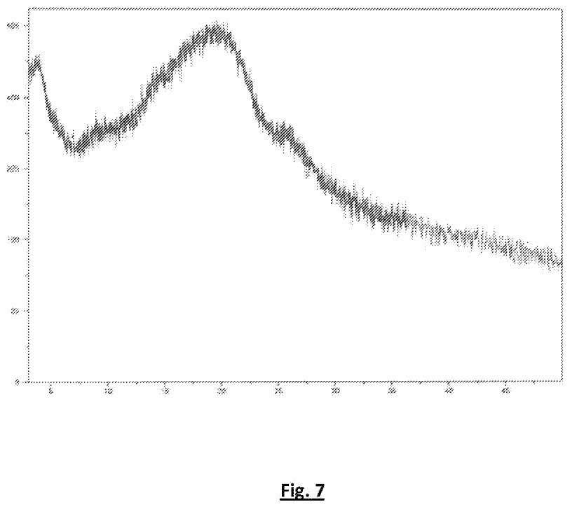

The solid was subjected to X-ray powder diffraction spectroscopy (XRPD) according to the method reported in the “Methods” section above, obtaining the spectrum shown in FIG. 7, which highlighted the obtaining of the amorphous form of voclosporin.

### Example 18—Process Step f

250 mg (0.206 mmol) of solid voclosporin obtained according to example 11 were dissolved in 1 ml MTBE (4 vol.).

After the addition of 10 ml (40 vol.) of cyclohexane, a white precipitate was formed. The solid was then filtered on a Buchner funnel to produce 25 mg of solid precipitate (10% yield). The solid was subjected to X-ray powder diffraction spectroscopy (XRPD) according to the method reported in the “Methods” section above, obtaining a spectrum completely similar to that shown in FIG. 7 and showing the obtaining of the amorphous form of voclosporin.

### Example 19—Stability Test of Voclosporin in Amorphous Form Obtained According to the Process of the Invention

Five 500 mg samples of amorphous voclosporin obtained according to Example 17 were prepared.

The first sample of reference, was subjected to X-ray powder diffraction spectroscopy (XRPD) according to the method reported in the “Methods” section above, obtaining a spectrum completely similar to that shown in FIG. 7 and highlighting the obtaining of the amorphous form of voclosporin.

The remaining four samples were instead tested for stability over time under different temperature, humidity, lighting, and time conditions as detailed below:
Test 1: the voclosporin sample was left in a sealed vial in a closed oven thermostated at 70° C. for 92 hours;
Test 2: the voclosporin sample was left in a sealed vial in a closed oven thermostated at 70° C. for 14 days;
Test 3: the voclosporin sample was left in a transparent vial in direct daylight for a period of 7 days; and
Test 4: the voclosporin sample was left in an open vial in an isolated environment with a saturated NaCl solution. The relative humidity reached 70% and the sample was left in these conditions for 7 days.

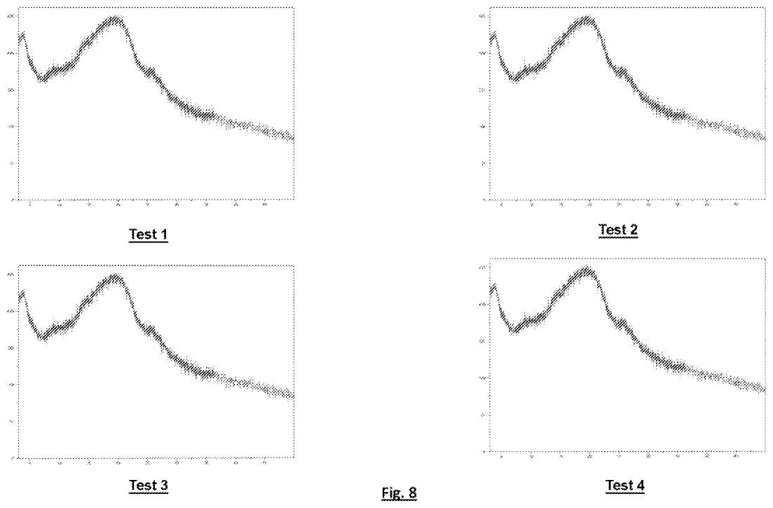

At the end of the tests, each sample was subjected to X-ray powder diffraction spectroscopy (XRPD) according to the method reported in the “Methods” section above, still obtaining a spectrum completely similar to that shown in FIG. 7 and highlighting the maintenance of the amorphous form of voclosporin. FIG. 8 shows the comparison of the amorphous forms as obtained from the analysis at the end of the stability tests.

1. A process for the preparation of voclosporin of formula (I) in amorphous form 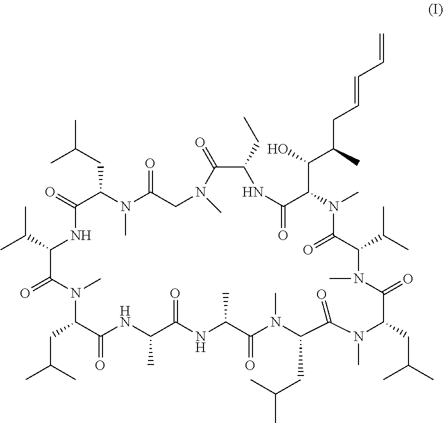 [SMILES: C=C/C=C/C[C@@H](C)[C@@H](O)[C@H]1C(=O)N[C@@H](CC)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]
comprising the steps of:
a. acetylating cyclosporine A in the presence of methyl tert-butyl ether (MTBE) with acetic anhydride in a stoichiometric amount with respect to cyclosporine A, thus obtaining acetyl cyclosporine A of formula II: 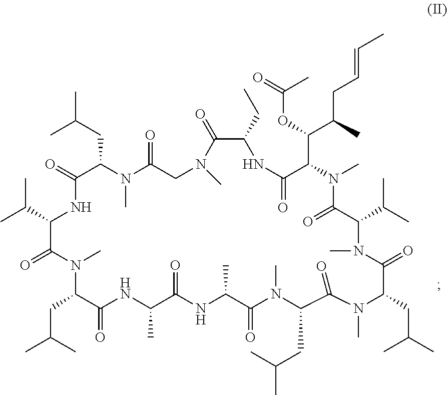 [SMILES: C/C=C/C[C@@H](C)[C@@H](OC(C)=O)[C@H]1C(=O)N[C@@H](CC)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]
b. oxidising the acetyl cyclosporine A of formula II with at least one oxidising agent that is an osmiate salt in the presence of a base selected from the group consisting of dimethylpyridine (or lutidine) and 2,4,6-trimethylpyridine (or sym-collidine), wherein said base is in an amount such that a pH value in the range from 6 to 8 is achieved, and in the presence of a co-oxidant selected from the group consisting of a periodate of an alkali or alkaline earth metal and a hypochlorite of an alkali or alkaline earth metal, thus obtaining acetyl cyclosporine A aldehyde of formula III: 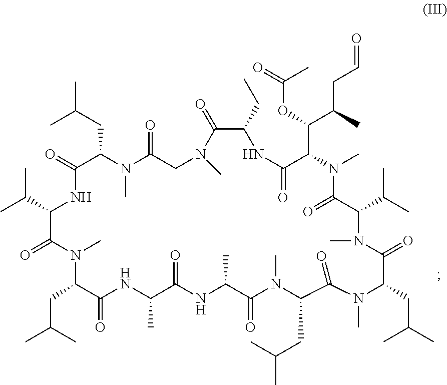 [SMILES: CC[C@@H]1NC(=O)[C@H]([C@H](OC(C)=O)[C@H](C)CC=O)N(C)C(=O)[C@H](C(C)C)N(C)C(=O)[C@H](CC(C)C)N(C)C(=O)[C@H](CC(C)C)N(C)C(=O)[C@@H](C)NC(=O)[C@H](C)NC(=O)[C@H](CC(C)C)N(C)C(=O)[C@H](C(C)C)NC(=O)[C@H](CC(C)C)N(C)C(=O)CN(C)C1=O]
c. converting the acetyl cyclosporine A aldehyde of formula III into acetyl voclosporin of formula IV: 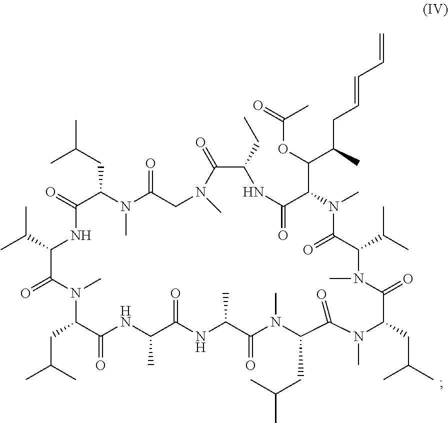 [SMILES: C=C/C=C/C[C@@H](C)C(OC(C)=O)[C@H]1C(=O)N[C@@H](CC)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]
d. crystallising the acetyl voclosporin of formula IV with a solvent system selected from the group consisting of THF/n-heptane and MTBE, thus obtaining acetyl voclosporin in crystalline form;
e. hydrolysing the acetyl voclosporin of formula IV, thus obtaining voclosporin of formula I; and
f. isolating the voclosporin of formula I obtained in step e. obtaining said voclosporin of formula I in amorphous form, wherein said isolation is carried out by precipitation with a solvent/anti-solvent system, wherein said solvent is selected from the group consisting of MTBE, THE, 2-methyl THF, diisopropyl ether, acetonitrile, acetone, ethanol and cyclopentylmethylether, and said anti-solvent is a hydrocarbon selected from the group consisting of normal heptane, cyclohexane and pentane.

2. The process according to claim 1, wherein the process comprises a step a1. subsequent to step a., which consist in crystallising acetyl cyclosporine A from a solvent system selected from the group consisting of MTBE/n-heptane and MTBE/cyclohexane.

3. The process according to claim 1, wherein in step b: the base is dimethylpyridine or lutidine.

4. The process according to claim 1, wherein said osmiate salt is an osmiate of an alkali or alkaline earth metal.

5. The process according to claim 1, wherein said step b. is conducted in the presence of the co-oxidant sodium periodate (NaIO₄).

6. The process according to claim 1, wherein in step d. the solvent system is THF/n-heptane.

7. The process according to claim 1, where in step d. the solvent system is MTBE.

8. A process for the preparation of acetyl cyclosporine A aldehyde of formula III: 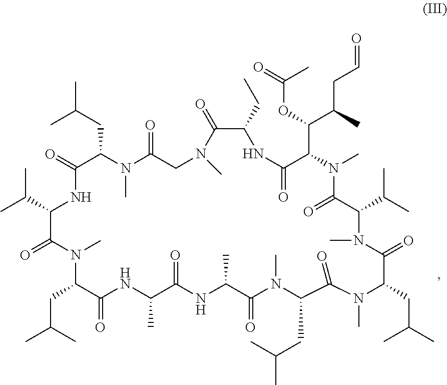 [SMILES: CC[C@@H]1NC(=O)[C@H]([C@H](OC(C)=O)[C@H](C)CC=O)N(C)C(=O)[C@H](C(C)C)N(C)C(=O)[C@H](CC(C)C)N(C)C(=O)[C@H](CC(C)C)N(C)C(=O)[C@@H](C)NC(=O)[C@H](C)NC(=O)[C@H](CC(C)C)N(C)C(=O)[C@H](C(C)C)NC(=O)[C@H](CC(C)C)N(C)C(=O)CN(C)C1=O]
comprising the steps of:
A. acetylating cyclosporine A in the presence of methyl tert-butyl ether (MTBE) with acetic anhydride in a stoichiometric amount with respect to cyclosporine A, thus obtaining acetyl cyclosporine A of formula II: 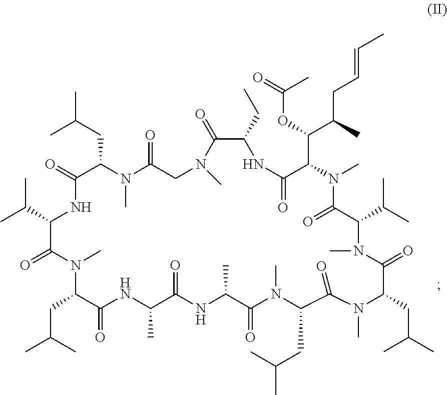 [SMILES: C/C=C/C[C@@H](C)[C@@H](OC(C)=O)[C@H]1C(=O)N[C@@H](CC)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]
and
B. oxidising the acetyl cyclosporine A of formula II with at least one oxidising agent which is an osmiate salt in the presence of a base selected from the group consisting of dimethylpyridine (or lutidine) and 2,4,6-trimethylpyridine (or sym-collidine), wherein said base is in an amount such that a pH value in the range from 6 to 8 is achieved, and in the presence of a co-oxidant selected from the group consisting of a periodate of an alkali or alkaline earth metal and a hypochlorite of an alkali or alkaline earth metal, thus obtaining acetyl cyclosporine A aldehyde of formula III.

9. The process according to claim 8, wherein the process comprises a step A1. following step A., which consists in crystallising acetyl cyclosporine A from a solvent system selected from the group consisting of MTBE/n-heptane and MTBE/cyclohexane.

10. A process for obtaining acetyl voclosporin of formula IV in crystalline form: 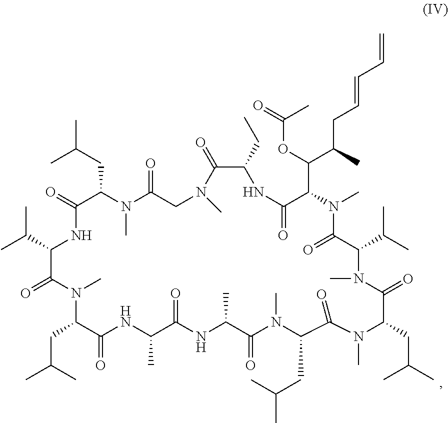 [SMILES: C=C/C=C/C[C@@H](C)C(OC(C)=O)[C@H]1C(=O)N[C@@H](CC)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]
comprising the steps of:
preparing acetyl voclosporin of formula IV;
crystallising said acetyl voclosporin with a solvent system selected from the group consisting of THF/n-heptane and MTBE, thus obtaining acetyl voclosporin IV.

11. A crystalline form of acetyl voclosporin of formula IV: 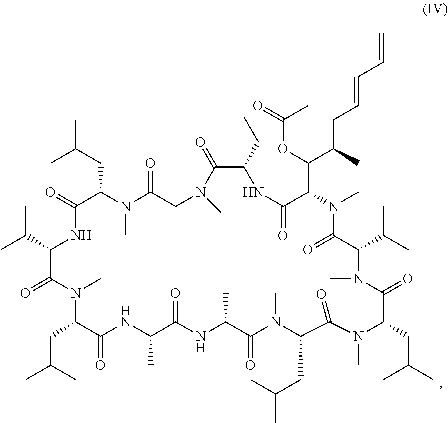 [SMILES: C=C/C=C/C[C@@H](C)C(OC(C)=O)[C@H]1C(=O)N[C@@H](CC)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]
wherein said crystalline form exhibits a X-ray powder diffraction spectrum (XRPD), with peaks at characteristic angle values 2ϑ(±0.2) of 5.0, 5.5, 7.0, 8.5, 8.8, 10.2, 12.4, 16.5, 17.2 and 20.9.

12. A process for obtaining an amorphous form of voclosporin of formula I 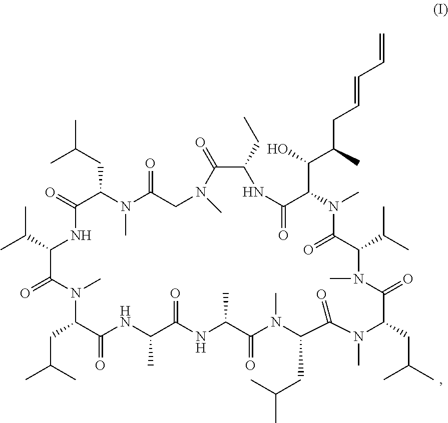 [SMILES: C=C/C=C/C[C@@H](C)[C@@H](O)[C@H]1C(=O)N[C@@H](CC)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)[C@@H](C(C)C)C(=O)N1C]
comprising the steps of:
preparing a solution of voclosporin of formula I in a solvent/antisolvent system, wherein said solvent is selected from the group consisting of MTBE, THF, 2-methyl THF, diisopropyl ether, acetonitrile, acetone, ethanol and cyclopentylmethylether and said antisolvent is a hydrocarbon selected from the group consisting of normal heptane, cyclohexane and pentane; and
recovering by precipitation from the solution thus prepared the amorphous form of voclosporin.

13. The process according to claim 4, wherein said osmiate salt is potassium osmiate K₂OsO₄.

14. The process according to claim 6, wherein THF/n-heptane is in a volume ratio of 3:8 to 5:8.

15. The process according to claim 6, wherein THF/n-heptane is in a volume ratio of 4:8.

16. The process according to claim 7, wherein MTBE is use in an amount from 30 to 40 volumes.

17. The process according to claim 7, wherein MTBE is use in an amount of about 35 volumes.
